(19) United States
(12) Patent Application Publication
(10) Pub. No.: US 2018/0079824 A1
(43) Pub. Date: Mar. 22, 2018

(54) HER2/ErbB2 Chimeric Antigen Receptor

(71) Applicants: Baylor College of Medicine, Houston, TX (US); Georg-Speyer-Haus, Frankfurt (DE)

(72) Inventors: Nabil M. Ahmed, Houston, TX (US); Stephen M. G. Gottschalk, Houston, TX (US); Winfried Wels, Frankfurt (DE)

(21) Appl. No.: 15/558,596

(22) PCT Filed: Mar. 18, 2016
(86) PCT No.: PCT/US16/23253

§ 371 Date: Sep. 15, 2017

Related U.S. Application Data
(60) Provisional application No. 62/135,014, filed on Mar. 18, 2015.

(51) Int. Cl. C07K 16/32; C07K 14/705; C12N 5/0783; A61K 35/17; A61K 45/06; C07K 14/725

(52) U.S. Cl. CPC C07K 16/32; C07K 14/70521; C12N 5/0636; A61K 35/17; C07K 2319/74; C07K 14/7051; C07K 2319/03; C07K 2317/622; C12N 2510/00; A61K 45/06

(57) ABSTRACT

Embodiments of the disclosure include immune cells expressing HER2-specific chimeric antigen receptors (CAR) and treatment of cancer therewith. In specific embodiments, sarcoma or glioblastoma are treated. In specific embodiments, such as for glioblastoma, for example, T-cells expressing a HER2-specific CAR are pp65CMV-specific T cells.

This application claims priority to U.S. Provisional Patent Application 62/135,014, filed Mar. 18, 2015, which is incorporated by reference herein in its entirety.

### TECHNICAL FIELD

The field of embodiments of the disclosure includes at least cell biology, molecular biology, immunology, oncology, and medicine.

### BACKGROUND

The HER2/ERBB2 antigen is identified with a variety of cancers, including at least breast, ovarian, lung, and brain, and its expression in the cancer cells is associated with poor prognosis for the individual.

Sarcomas are also associated with HER2/ERBB2 antigen and are a diverse group of malignancies that include osteosarcoma (OS), Ewing's sarcoma (EWS), rhabdomyosarcoma (RMS), and non-rhabdomyosarcoma soft tissue sarcomas (NRSTS), such as synovial sarcoma or desmoplastic small round cell tumors. While patients with local disease have an excellent outcome, the prognosis of patients with advanced stage disease remains poor. Cell therapy in the form of high dose chemotherapy with autologous stem cell rescue has been extensively explored for sarcomas. However, most studies have not shown a significant survival benefit in comparison to standard chemotherapy, indicating that more specific cell therapies are needed to improve outcomes.

Immunotherapy with antigen-specific T cells may benefit sarcoma patients since immune-mediated killing does not rely on pathways employed by conventional therapies to which such tumors are often resistant. Adoptive transfer of T cells, genetically modified to express chimeric antigen receptors (CARs), has shown great promise in early phase clinical studies for the therapy of CD19-positive malignancies. Clinical experience using this approach for solid tumors, however, is much more limited. CARs recognize antigens expressed on the cell surface of tumor cells, and several potential CAR target antigens have been identified for sarcoma, including human epidermal growth factor receptor 2 (HER2), GD2, interleukin (IL)11Rα, and B7H3. Aliases for HER2 include HER2/neu, NEU, V-Erb-B2 avian erythroblastic leukemia viral oncogene homolog 2, V-Erb-B2, receptor tyrosine-protein kinase ErbB-2, proto-oncogene C-ErbB2, ErbB2, Neuroblastoma/Glioblastoma derived oncogene homolog, CD340, TKR1, p185erB2, MLN19, EC2.7.10.1, EC 2.7.10 (http://www.genecards.org/cgi-bin/carddisp.p1?gene=ERBB2). Although sarcoma cells are often HER2-positive, the HER2 gene locus is not amplified in this disease. Thus sarcomas belong to a large group of malignancies that includes cancers of the lung, ovary, prostate, and brain, which express HER2 at levels too low for HER2 monoclonal antibodies to be effective.

Even malignancies that express HER2 at low levels can be targeted with T cells that express HER2-specific CARs (HER2-CAR T cells). These HER2-CAR T cells kill both “bulk” tumor cells and tumor-initiating cells (which have been shown to express HER2 at 3-5 fold the bulk tumor expression levels) and have potent antitumor activity in preclinical animal models.

There is a need in the art to provide safe and effective cell therapy to individuals with any type of HER2-positive cancers.

### BRIEF SUMMARY

Embodiments of the disclosure concern methods and compositions for treating and prevention of HER2-positive cancers. The HER2-positive cancer may be of any kind, including brain tumors (for example, but not limited to, glioblastoma, medulloblastoma, ependymoma and metastatic deposits and/or infiltrates from HER2-positive cancer outside the neuraxis), sarcoma, breast cancer, ovarian cancer, stomach cancer, uterine cancer, endometrial cancer, lung cancer, prostate cancer, and so forth. In cases wherein the individual has sarcoma, the type may be soft tissue sarcoma or osteosarcoma, for example. The sarcoma may be of any subtype, including angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, or synovial sarcoma, for example. In specific embodiments, the cancer is recurrent and/or refractory. The cancer may or may not have metastasized. The cancer may be present in the individual as a solid tumor. The individual may be an infant, child, adolescent, or adult of any gender.

Embodiments of the disclosure encompass immune cells that express a HER2-targeting chimeric antigen receptor (CAR) and uses thereof. In certain aspects, the CAR comprises a scFv specific for HER2, or any natural or artificial moiety that can specifically bind HER2/ERBB2. In particular embodiments, the CAR utilizes a single chain variable fragment (scFv) specific for HER2 that is known in the art, although in other embodiments, the CAR does not utilize an scFv specific for HER2 that is known in the art. In certain embodiments, the CAR utilizes an scFv specific for HER2 that is derived from a monoclonal antibody known in the art, whereas in other cases the CAR utilizes an scFv specific for HER2 that is not derived from a monoclonal antibody known in the art. The CAR may be a second generation or third generation CAR, but in specific embodiments the CAR is not a third generation CAR and comprises only one costimulatory endodomain.

In specific embodiments, an individual is given a certain type of immunotherapy for treating and preventing HER2-positive cancer, such as an immune cell that recognizes HER2. In specific embodiments, the immune cell is a T cell, NK cell, or NKT cell. Other effector cells include those that can exhibit antitumor activities either innately or are modified to exhibit this effect. In specific embodiments the immune cells comprise HER2-specific CAR and may be further modified other than the HER2-specific CAR. In specific embodiments, the HER2-specific CAR comprises a scFv derived from trastuzmab, FRP5, 800E6, F5cys, pertuzumab or a combination thereof, for example. Another genetic modification of the immune cells is to express one or more chemokine receptors, such that they are utilized to enhance T-cell homing to tumor sites, for example. In specific embodiments of the CAR T cells, one can transgenically express one or more stimulatory cytokines. In certain embodiments, one can render HER2-CAR T cells resistant to an inhibitory tumor microenvironment. One may also avoid ‘on target/off cancer’ toxicity with genetic modifications to increase safety, such as an inducible suicide gene (such as caspase-9, for example) and/or inhibitory receptors to limit the effector function of T cells to tumor sites.

In particular embodiments, an individual in need thereof, such as one that is known to have a HER2-positive cancer or suspected of having a HER2-positive cancer, is provided a therapeutically effective amount of immune cells encompassed by the disclosure. In particular embodiments, an individual may be given between 1×10⁴/m² and 1×10¹⁰/m² HER2-CAR T cells in a given administration, although other doses may be utilized. Multiple administrations of cells may be provided to the individual. In certain embodiments, one does or does not use lymphodepleting chemotherapy or irradiation prior to T-cell transfer. In particular embodiments, there is no post-therapy infusion with a cytokine, such as IL2, although in alternative embodiments there is post-therapy infusion with a cytokine. In specific embodiments, one can combine HER2-CAR immune cells with one or more additional immunological cancer therapies, such as checkpoint antibodies, immune modulating agents, or vaccines to increase T-cell activation and prolong in vivo survival. Other cancer therapies may also be used, such as surgery, radiation, drug therapy, and/or hormone therapy, for example.

In one embodiment, there is a polynucleotide that encodes a HER2 chimeric antigen receptor, and the chimeric antigen receptor may comprise a transmembrane domain selected from the group consisting of CD3-zeta, CD28. CD8, 4-1BB, CTLA4, CD27, and a combination thereof. In some embodiments, the chimeric antigen receptor comprises no more than one costimulatory endodomain, although in certain embodiments the chimeric antigen receptor comprises more than one costimulatory endodomain. In particular embodiments, the chimeric antigen receptor comprises co-stimulatory molecule endodomains selected from the group consisting of CD28, CD27, 4-1BB, OX40 ICOS, Myd88, CD40, and a combination thereof. The chimeric antigen receptor may comprise a scFv specific for HER2 that is selected from the group consisting of trastuzmab, FRP5, scFv800E6, F5cys, pertuzumab and a combination thereof.

In a certain embodiment, there is an expression vector comprising a polynucleotide encompassed by the disclosure, and the vector may be a viral vector, such as a retroviral vector, lentiviral vector, adenoviral vector, or adeno-associated viral vector, or it may be a non-viral vector.

In a particular embodiment, there is a cell comprising a polynucleotide or expression vector as encompassed by the disclosure. In specific embodiments, the cell is an immune cell, such as a T cell, NK cell, or NKT cell. The cell may be specific for another antigen, including a tumor antigen in some cases. In specific embodiments, the cells are pp65CMV-specific T cells, CMV-specific T cells, EBV-specific T cells, Varicella Virus-specific T cells, Influenza Virus-specific T cells and/or Adenovirus-specific T cells.

In one embodiment, there is a method of treating an individual for cancer, comprising the step of providing to the individual a therapeutically effective amount of a plurality of any of the cells as encompassed by the disclosure. In specific embodiments, the cancer is HER2 positive. The cancer may be refractory or recurrent. In specific embodiments, the cancer is sarcoma or glioblastoma. The sarcoma may be osteosarcoma, for example. Doses may be formulated otherwise, such as per weight or per age. In certain embodiments, the therapeutically effective amount of a plurality of the cells is at a dose of at least 1×10⁴/m², 1×10⁵/m², 1×10⁶/m², 1×10⁷/m², 1×10⁸/m², 1×10⁹/m², or 1×10¹⁰/m². In specific embodiments, the therapeutically effective amount of a plurality of the cells is at a dose of no more than 1×10¹⁰/m², 1×10⁹/m², 1×10⁸/m², 1×10⁷/m², 1×10⁶/m², 1×10⁵/m², or 1×10⁴/m². In particular embodiments, the method occurs without or with the administration of one or more cytokines and without or with lymphodepleting therapy and occurs with a cell dose in the range of 1×10⁴/m² to 1×10¹⁰/m². The cytokine may be IL2, IL7, IL12, and/or IL15.

In particular embodiments, the use of immune cells expressing HER2-specific chimeric antigen receptors occurs ex vivo. For example, the immune cells may be exposed ex vivo to one or more cells, one or more tissues and/or one or more organs for the cells to target HER2-bearing cells, including HER2-expressing cancer cells, In a specific embodiment, the HER2-specific chimeric antigen receptor-expressing immune cells are utilized to process one or more cells, one or more tissues and/or one or more organs ex vivo. In particular examples, one can purge tissue(s) or organ(s) from some or all HER2-expressing cancer cells by exposing ex vivo an effective amount of the HER2-specific chimeric antigen receptor-expressing immune cells to the respective tissue(s) or organ(s). As one example, bone marrow can be exposed ex vivo to the HER2-specific chimeric antigen receptor-expressing immune cells prior to transplant or HER2-specific chimeric antigen receptor-expressing immune cells can be used for processing an organ that harbors a malignancy prior to introduction into a host in need thereof.

Methods of generating immune cells that express a HER2-specific chimeric antigen receptor are contemplated herein. In specific embodiments, immune cells to be manipulated to express a HER2-specific chimeric antigen receptor are obtained from another party, including commercially or a skilled artisan, or are isolated from an individual to be treated with the HER2-specific chimeric antigen receptor immune cell or are isolated from another individual. The immune cell may modified to express the HER2-specific chimeric antigen receptor using standard means in the art, such as upon transduction of a polynucleotide that encodes the HER2-specific chimeric antigen receptor. In cases wherein the obtained or isolated immune cell is genetically modified to express the HER2-specific chimeric antigen receptor and also comprises a second genetic modification (such as expresses another chimeric antigen receptor or another type of non-natural receptor), the order in which the genetic modifications can occur may be in any order. Any polynucleotide that encodes a HER2-specific chimeric antigen receptor or another type of receptor may be transduced into the cell using a vector, such as a viral or non-viral vector. A viral vector may be a retroviral, lentiviral, adenoviral, adeno-associated viral vector, and so forth.

In specific embodiments, a cell is an immune cell that transgenically expresses one or more chemokine receptors, such as wherein the chemokine receptor is a receptor for a chemokine expressed by the cancer. In specific embodiments, the chemokine is CXCL1, CXCL8, CCL2, and/or CCL17. An individual may be provided a therapeutically effective amount of an additional cancer therapy, such as one given to the individual before, during, and/or after the individual is given the plurality of cells. In specific embodiments, the additional therapy comprises surgery, drug therapy, chemotherapy, radiation, immunotherapy, or a combination thereof. In specific embodiments, the individual is given lymphodepleting therapy prior to being given the plurality of cells, although in some embodiments the individual is not given lymphodepleting therapy prior to being given the plurality of cells.

In certain embodiments, the immunotherapy comprises one or more checkpoint antibodies, such as checkpoint antibodies that recognize CTLA4, PD-1, PD-L1, TIM3, BLTA, VISTA and/or LAG3. In particular embodiments, the cell comprises an inhibitory receptor.

In an embodiment, there is a kit, comprising a polynucleotide as encompassed by the disclosure, an expression vector as encompassed by the disclosure, and/or cells as encompassed by the disclosure, wherein the polynucleotide, expression vector, and or cells are housed in a suitable container.

In certain embodiments, there are HER2 chimeric antigen receptor modified CMV-specific T-cells for use in cancer. Although they may be employed for any individual with any type of cancer, in specific embodiments they are utilized for progressive glioblastoma, for example.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1: Plasma cytokine levels post HER2-CAR T-cell infusion. Plasma cytokine levels post HER2-CAR T-cell infusion were measured by multiplex analysis. Results for IFNγ, TNFα, IL6, and IL8 are shown. There was a significant increase of plasma IL8 levels at 1 (p=0.028), 2 (p=0.006), and 4 (p=0.001) weeks post T-cell infusion. Results for GM-CSF, IL1β, IL2, IL4, IL5, IL7, IL10, IL12p70, and IL13 are shown in FIGS. 6A and 6B.

FIGS. 2A-2F: In vivo persistence of HER2-CAR T cells. (2A-2C) In vivo persistence of T cells at each dose level (DL). (2D) Correlation of cell dose and level of transgene detection 3 hours post HER2-CAR T-cell infusion. (2E) Detection of HER2-CAR T cells 6 weeks post infusion was dependent on the infused T cell dose (≦1×10⁶/m² vs >1×10⁶/m²: p=0.002). (2F) HER2-CAR T cells were detected for up to 18 months post infusion.

FIGS. 3A and 3B: HER2-CAR T-cell homing to tumor sites. (3A) Immunohistochemistry for CD3 expression in tumor biopsy. (3B) Transgene detection in tumor biopsy and corresponding peripheral blood sample.

FIGS. 4A, 4B, and 4C: Outcome post HER2-CAR T-cell infusion. (4A) Kaplan-Meier curve of all infused patients (n=19). (4B) Prominent necrosis (P14) of tumor after HER2-CAR T-cell infusion. (4C) PET images (P4) before and 6 weeks after HER2-CAR T-cell infusion.

FIGS. 5A, 5B, and 5C: Characterization of HER2-CAR T-cell product. (5A) HER2-CAR expression on non-transduced (NT) and transduced. T cells. NT vs HER-CAR T cells, p<0.0001). Individual data points and mean is shown. (5B) Phenotypic analysis of HER2-CAR T-cell product. CM: central memory (CD3+/CD45RO+/CD62L+); EM: effector memory (CD3+/CD45RO+/CCR7−/CD62L−). Box plot with whiskers (Tukey method) is shown. (5C) Cytotoxicity assay using NT- and HER2-CAR T cells as effectors and HER2-negative (K562, MDA-MB-468 (MDA)), or HER2-positive (NCI-H1299, LM7) cell lines as targets. Mean with standard deviation at an effector to target ratio of 20:1 is shown. K562: NT vs HER2-CAR T cells, p=NS; MDA: NT vs HER2-CAR T cells, p=NS; NCI-H1229: NT vs HER2-CAR T cells, p<0.0001; LM7: NT vs HER2-CAR T cells, p<0.0001.

FIGS. 6A and 6B: Plasma cytokine levels post HER2-CAR T-cell infusion. Plasma cytokine levels post HER2-CAR T-cell infusion were measured by multiplex analysis. Results for GM-CSF, IL1β, IL2, IL4, IL5, IL7, IL10, IL12p70, and IL13 are shown here. There were no significant changes post T-cell infusion. Results for IFNγ, TNFα, IL6, and IL8 are shown in FIG. 1.

FIG. 7: In vivo persistence of T cells. Six patients received at least two doses of HER2-CAR T cells (Shown for patients 5, 7, 12, 14, and 18). HER2-CAR T cells were detected by qPCR post infusion. The pattern of HER2-CAR T-cell persistence was similar after both infusions. Right panel shows the data with a y-axis max of 250 copies per mg DNA and left panel with a y-axis max of 50 copies per mg DNA.

FIGS. 8A-8C: In vivo persistence of human epidermal growth factor receptor 2 (HER2) chimeric antigen receptor (CAR)/cytomegalovirus (CMV)-specific T-cells in patients with progressive glioblastoma. (8A) In vivo persistence as detected by qPCR at each dose level. (8B) Detection of HER2-CAR T-cells in the peripheral blood in patients receiving 2 or more infusions. (8C) HER2-CAR transgene was detected for up to 12 months after T-cell infusion.

FIGS. 9A-9C: Clinical outcome in patients with glioblastoma after intravenous infusion of human epidermal growth factor receptor 2 (HER2) chimeric antigen receptor (CAR)/cytomegalovirus (CMV)-specific T-cells. (9A) Magnetic resonance imaging (MRI) of the brain before and 6 weeks after HER2/CMV T-cell infusion. (9B) Swimmer's plot showing the disease status and survival in all patients treated with HER2/CMV T cells. (9C) Kaplan-Meier curve of all infused patients (n=17) showing the overall survival (OS) from first infusion (upper left panel), OS from diagnosis (upper right panel), time to progression (TTP) from first infusion and survival according to prior salvage therapy.

FIG. 10: Detection of HER2, CMV pp65, and CMV IE1 expression by immunohistochemistry in GBMs of study patients. Immunohistochemisty was used to detect HER2, CMV pp65, and CMV IE1 expression. Results were graded according to the following scheme: Intensity: 0 to 3+ based on positivity of control slides; Grade (percentage of positive tumor cells): 0=none, 1=1-25%, 2=26-50%, 3=51-75%, 4=76-100%. Representative images are shown (magnification 100-fold). Results for all patients are summarized in Table 5.

FIGS. 11A-11D: Characterization of HER2/CMV T-cell product. (11A) HER2-CAR expression on non-transduced (NT) and transduced. T cells. NT vs HER-CAR T cells, p<0.0001). Individual data points and mean is shown. (11B) Phenotypic analysis of HER2/CMV T-cell product. CM: central memory (CD3+/CD45RO+/CD62L+); EM: effector memory (CD3+/CD45RO+/CCR7−/CD62L−). Box plot with whiskers (10 to 90 percentile) is shown. (11C) Cytotoxicity assay using CMV and HER2/CMV T cells as effectors and HER2-negative (K562, autologous (auto) or HLA mismatched (MM) LCL), or HER2-positive (U373) cell lines as targets. Mean with standard deviation at an effector to target ratio of 20:1 is shown. LCL-MM: CMV vs HER2/CMV T cells, p=NS; LCL-Auto: CMV vs HER2/CMV, p=NS; K562: CMV vs HER2/CMV T cells, p=NS; U373: CMV vs HER2/CMV T cells, p<0.0001; LCL-MM vs LCL-auto: for CMV and CMV/HER2-T cells: p<0.001). (11D) Antigen-specificity HER2/CMV T-cell product was determined by IFN-γ Elispot assays using CMV pp65, CMV IE1, and hexon/penton (Adv) pepmixes, and auto-LCL as stimulators. PHA served as positive control (pos Co) and media as negative control (neg Co). Box plot with whiskers (10 to 90 percentile) is shown; p<0.001 for CMV pp65 vs CMV IE1, CMV pp65 vs CMV IE1, CMV pp65 vs Adv, CMV pp65 vs Auto-LCL, CMV pp65 vs neg Co, and Auto-LCL vs IE1; p<0.005 for Adv vs CMV IE1.

FIG. 12: Precursor frequency of CMV-, Adv-, and EBV-specific T-cells post HER2/CMV T-cell infusion. Blood samples were obtained Pre, and 1, 2, 4, and 6 week (wk) post T-cell infusion. The frequency of CMV pp65-, CMV IE1-, Adv-, and EBV-specific T cells was determined by IFN-γ Elispot assays using pepmixes (CMV pp65, CMV IE1, Adv hexon/penton) or auto-LCL as stimulators. Individual patients (dotted lines) and mean (solid line) is shown. No significant differences were observed between individual time points detailed description

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein embodiments which are disclosed and still obtain a like or similar result without departing from the spirit and scope of the invention.

Embodiments of the disclosure concern treatment or prevention of any type of cancer. For example, the outcome for patients with metastatic or recurrent sarcoma remains poor. Adoptive therapy with tumor-directed T cells is an attractive therapeutic option, but has never been evaluated in sarcoma. A study described herein was conducted in which patients with recurrent/refractory HER2-positive sarcoma received certain escalating doses (1×10⁴/m² to 1×10⁸/m²) of T cells expressing a HER2-specific chimeric antigen receptor with a CD28.ζ signaling domain (HER2-CAR T cells in which the CAR contained an ectodomain derived from the HER2-specific MAb FRP5). In specific embodiments, an ultra-low dose of HER2-CAR T cells (1×10⁴/m²) as a single agent without the administration of IL2 or lymphodepleting chemotherapy was employed, and the cell dose was escalated to 1×10⁸/m². The present disclosure demonstrates the safety, persistence and antitumor activity of the infused cells.

In other embodiments of the disclosure, the HER2-specific CARs are utilized for glioblastoma (GBM). As described herein, adoptive immunotherapy with HER2-specific chimeric antigen receptor (CAR)-modified CMV-specific T-cells was utilized for GBM. As provided herein, CMV-seropositive patients with recurrent/progressive HER2-positive GBM received autologous T cells specific for the CMV antigen pp65 that were genetically modified to express a HER2-CAR with a CD28.ζ signaling domain (HER2/CMV T-cells). As examples, multiple adult and pediatric patients with recurrent/progressive HER2-positive GBM received one or more infusions of 1×10⁶/m² to 1×10⁸/m² HER2/CMV T-cells. T-cell infusions were well tolerated with no dose limiting toxicities. HER2/CMV T-cells were detected in the peripheral blood for up to 12 months post-infusion as judged by real-time qPCR. Of 16 evaluable patients, 1 patient had a partial response for >9 months, 7 patients had stable disease (SD) for 2•3 to >30 months, and 8 patients progressed after T-cell infusion. Three patients with SD are currently alive without any evidence of progression at >30 months of followup. For the entire study cohort, the median OS was 11•6 months from the first T-cell infusion and 24 8•months from diagnosis. In particular embodiments, HER2/CMV T-cells may be utilized as a single agent or in combination with other immunomodulatory approaches for GBM.

### II. Chimeric Antigen Receptors

Genetic engineering of immune cells (such as human T lymphocytes) to express tumor-directed chimeric antigen receptors (CAR) can produce antitumor effector cells that bypass tumor immune escape mechanisms that are due to abnormalities in protein-antigen processing and presentation. Moreover, these transgenic receptors can be directed to tumor-associated antigens that are not protein-derived. In certain embodiments of the invention there are CTLs that are modified to comprise at least one CAR. In specific embodiments, a single immune cell expresses one type of CAR molecule, or a single cell may express multiple types of CAR molecules and/or other non-natural receptors, such as T cell receptors, chemokine receptors, or α/β receptors.

In particular cases, the cytotoxic T lymphocytes (CTLs) include a receptor that is chimeric, non-natural and engineered at least in part by the hand of man. In particular cases, the engineered CAR has one, two, three, four, or more components, and in some embodiments the one or more components facilitate targeting or binding of the T lymphocyte to the tumor antigen-comprising cancer cell. In specific embodiments, the CAR comprises an antibody for the tumor antigen, part or all of a cytoplasmic signaling domain, and/or part or all of one or more co-stimulatory molecules, for example endodomains of co-stimulatory molecules. In specific embodiments, the antibody is a single-chain variable fragment (scFv). In certain aspects the antibody is directed at HER2 (which is also called receptor tyrosine-protein kinase erbB-2, also known as CD340 (cluster of differentiation 340), proto-oncogene Neu, or ERBB2 (human)), for example. In specific embodiments, a single CAR molecule is bi-specific for two antigens by comprising a CAR that comprises a scFv that targets HER2 and also comprises another scFv that targets an antigen other than HER2.

In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD28, CD27, 4-1BB (CD137), OX40 (CD134), ICOS, Myd88, and/or CD40. In particular embodiments, co-stimulatory molecules are employed to enhance the activation, proliferation, and cytotoxicity of T cells produced by the CAR after antigen engagement. T-cells can also be further genetically modified to enhance their function. Examples, but not limited to, include the transgenic expression of cytokines (e.g. IL2, IL7, IL15), silencing of negative regulators (for example SHP-1, FAS, PD-L1), chemokine receptors (e.g. CXCR2, CCR2b), dominant negative receptors (e.g. dominant negative TGFβRII), and/or so called ‘signal converters’ that convert a negative into a positive signal (e.g. IL4/IL2 chimeric cytokine receptor, IL4/IL7 chimeric cytokine receptor, or TGFβRII/TLR chimeric receptor).

In a particular embodiment, the components of the CAR in the polynucleotide that encodes it are in a particular order so that the expressed CAR protein has the corresponding domains in a particular order. For example, in particular embodiments the transmembrane domain is configured between the antibody domain and the endodomain. In specific embodiments, the order of the domains in the encoded CAR protein is N-terminal-antibody-transmembrane domain-endodomain-C terminal, although in certain cases the order of the domains in the encoded CAR protein is N-terminal-endodomain-transmembrane domain-antibody-C terminal. Of course, other domains may be inserted within this configuration, with care being taken to place it on the appropriate side of the transmembrane domain to be located inside the cell or on the surface of the cell. Those domains that need to be intracellular need to be on the flank of the transmembrane domain in the protein that the endodomain is located, for example. Those domains that need to be extracellular need to be on the flank of the transmembrane domain in the protein that the antibody is located.

The CAR may be first generation (CAR that includes the intracellular domain from the CD3 ξ-chain), second generation (CAR that also includes intracellular signaling domains from various costimulatory protein receptors (e.g., CD28, 41BB, ICOS)), or third generation (CAR in which there are multiple signaling domains, such as when signaling is provided by CD3-ζ together with co-stimulation provided by CD28 and a member of the tumor necrosis factor receptor superfamily, such as 4-1BB or OX40), for example. In specific embodiments the CAR comprises a single costimulatory domain, however.

The CAR may be specific for HER2. Cells expressing HER2-specific CARs may additionally express one or more additional CARs, e.g., CARs that bind to a TAA or TSA, e.g., such as those specific for EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE AI, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, VEGF receptors, and/or other exemplary antigens that are present with in the extracelluar matrix of tumors, such as oncofetal variants of fibronectin, tenascin, or necrotic regions of tumors and other tumor-associated antigens or actionable mutations that are identified through genomic analysis and or differential expression studies of tumors, for example.

In certain embodiments, a CAR that directs an immune cell to HER2 comprises (1) an extracellular antigen-binding domain that binds to HER2, and (2) an intracellular domain that comprises a primary signaling moiety, e.g., a CD3ζ chain, that provides a primary T cell activation signal, and optionally a costimulatory moiety, e.g., a CD28 polypeptide and/or a 4-1BB (CD137) polypeptide.

In particular cases, the CAR is specific for HER2, and in certain embodiments, the present invention provides chimeric T cells specific for HER2 by joining an extracellular antigen-binding domain derived from the HER2-specific antibody to cytoplasmic signaling domains derived from the T-cell receptor ζ-chain, with the endodomains of the exemplary costimulatory molecules CD28 and OX40, for examples. This CAR is expressed in human T cells and the targeting of HER2-positive cancers is encompassed in the invention. In some cases, the same cell comprises a CAR specific for HER2 and a CAR specific for another tumor antigen.

In particular embodiments, a CAR specific for HER2 refers to a CAR having a scFv antibody that recognizes HER2. Although in some embodiments the HER2 scFv is of any kind, in other embodiments the scFv is derived from MAbs selected from the group consisting of trastuzmab, FRP5, 800E6, F5cys, pertuzumab and a combination thereof.

In specific embodiments, a representative HER2 nucleotide sequence is at the National Center for Biotechnology Institute's GenBank® database at Accession No. NM_004448, which encodes a protein such as is at Accession No. NP_004439, both of which are incorporated by reference herein in their entirety. A skilled artisan recognizes how to manipulate a HER2 protein sequence to generate monoclonal antibodies to be utilized in HER-2 specific CARS as encompassed by the disclosure.

Although in particular embodiments the HER2 CAR is expressed from an immune cells, in other embodiments the HER2 CAR is provided to the individual on a substrate. The substrate may be of any kind so long as it is biocompatible and one or more HER2 CAR molecules are suitably affixed thereto. In specific cases, the substrate is not a cell comprises exosomes microsomes, micelles, or artificial bodies, such as nanoparticles, beads, and so forth. Providing an effective amount of HER2 CAR-comprising substrate(s) to the individual may be utilized as a single therapy, or they may be delivered to an individual in need thereof in addition to HER 2 CAR-expressing immune cells and/or another therapy (drug, immunotherapy, surgery, radiation, etc.). The coated substrates can elicit an antitumor response. CAR molecules can be introduced as an encoding transgene and these cell products harvested from an expressing cell or cell line. Alternatively, these CAR molecules can be secreted and physically introduced to miscelles or liposomes.

### III. Host Cells Expressing HER2 CAR

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T-cell (also known as TC, Cytotoxic T Lymphocyte, CTL, T-Killer cell, cytolytic T cell, CD8+ T-cells, CD4+ T-cells, or killer T-cells); NK cells and NKT cells are also encompassed in the invention. Bacterial cells, such as E. coli, may be employed to generate the polynucleotide that encodes the HER2-CAR, for example.

In one aspect, provided herein is a cell that has been genetically engineered to express one or more CARs. In certain embodiments, the genetically engineered cell is, e.g., a T lymphocyte (T-cell), a natural killer (NK) T-cell, or an NK cell. In certain other embodiments, the genetically engineered cell is a non-immune cell, e.g., a mesenchymal stem cell (MSC), a neuronal stem cell, a hematopoietic stem cell, an induced pluripotent stem cell (iPS cell), or an embryonic stem cell, for example. In specific embodiments, the cell also comprises an engineered CAR or any other genetic modification that may enhance its function. In a particular embodiment, the antigen binding domain of the CAR binds HER2, although in certain embodiments the antigen binding domain of a CAR recognizes a different target antigen.

In certain embodiments, it is contemplated that RNAs or proteinaceous sequences may be co expressed with other selected RNAs or proteinaceous sequences in the same cell, such as the same CTL. Co expression may be achieved by co transfecting the CTL with two or more distinct recombinant vectors. Alternatively, a single recombinant vector may be constructed to include multiple distinct coding regions for RNAs, which could then be expressed in CTLs transfected with the single vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the genetically engineered T-cells, where one wishes to terminate the treatment, the cells become neoplastic, in research where the absence of the cells after their presence is of interest, or other purpose. For this purpose one can provide for the expression of certain gene products in which one can kill the engineered cells under controlled conditions, such as inducible suicide genes. Such suicide genes are known in the art, e.g., the iCaspase9 system in which a modified form of caspase 9 is dimerizable with a small molecule, e.g., AP1903. See, e.g., Straathof et al., Blood 105:4247-4254 (2005).

It is further envisaged that the pharmaceutical composition of the disclosure comprises a host cell transformed or transfected with a vector defined herein. The host cell may be produced by introducing at least one of the above described vectors or at least one of the above described nucleic acid molecules into the host cell. The presence of the at least one vector or at least one nucleic acid molecule in the host may mediate the expression of a gene encoding the above described be specific single chain antibody constructs.

The described nucleic acid molecule or vector that is introduced in the host cell may either integrate into the genome of the host or it may be maintained extrachromosomally.

The host cell can be any prokaryote or eukaryotic cell, but in specific embodiments it is a eukaryotic cell. In specific embodiments, the host cell is a bacterium, an insect, fungal, plant or animal cell. It is particularly envisaged that the recited host may be a mammalian cell, more preferably a human cell or human cell line. Particularly preferred host cells comprise immune cells, CHO cells, COS cells, myeloma cell lines like SP2/0 or NS/0.

The pharmaceutical composition of the disclosure may also comprise a proteinaceous compound capable of providing an activation signal for immune effector cells useful for cell proliferation or cell stimulation. In the light of the present disclosure, the “proteinaceous compounds” providing an activation signal for immune effector cells may be, e.g. a further activation signal for T-cells (e.g. a further costimulatory molecule: molecules of the B7-family, OX40 L, 4-1BBL), or a further cytokine: interleukin (e.g. IL-2, IL-7, or IL-15), or an NKG-2D engaging compound. The proteinaceous compound may also provide an activation signal for immune effector cell, which is a non-T-cell. Examples for immune effector cells which are non-T-cells comprise, inter alia, NK cells, or NKT-cells.

One embodiment relates to a process for the production of a composition of the disclosure, the process comprising culturing a host cell defined herein above under conditions allowing the expression of the construct, and the cell or a plurality of cells is provided to the individual.

The conditions for the culturing of cells harboring an expression construct that allows the expression of the CAR molecules are known in the art, as are procedures for the purification/recovery of the constructs when desired.

In one embodiment, the host cell is a genetically engineered T-cell (e.g., cytotoxic T lymphocyte) comprising a CAR and in particular embodiments the cell further comprises an engineered TCR. Naturally occurring T-cell receptors comprise two subunits, an α-subunit and a β-subunit, each of which is a unique protein produced by recombination event in each T-cell's genome. Libraries of TCRs may be screened for their selectivity to particular target antigens. An “engineered TCR” refers to a natural TCR, which has a high-avidity and reactivity toward target antigens that is selected, cloned, and/or subsequently introduced into a population of T-cells used for adoptive immunotherapy. In contrast to engineered TCRs, CARs are engineered to bind target antigens in an MHC independent manner.

In specific embodiments, the cell is an immune cell that transgenically expresses one or more chemokine receptors including wherein the chemokine receptor is a receptor for a chemokine expressed by the cancer. In certain cases, the chemokine is CXCL1, CXCL8, CCL2, and/or CCL17.

Additional engineering of the CAR T cells themselves may comprise using transgenic expression of stimulatory cytokines, or by rendering HER2-CAR T cells resistant to the inhibitory tumor microenvironment. For example transgenic expression of cytokines, such as IL15, renders T cells resistant to the inhibitory effects of regulatory T cells (Tregs). Alternatively, transgenic expression of IL12 in CAR T cells reverses the immunosuppressive tumor environment by triggering the apoptosis of inhibitory tumor-infiltrating macrophages and myeloid derived suppressor cells (MDSCs). Conversely, instead of being engineered to produce cytokines, CAR T cells can be engineered to be resistant to cytokines that inhibit their cytolytic function. TGFβ is widely used by tumors as an immune evasion strategy, since it promotes tumor growth, limits effector T-cell function, and activates Tregs. These detrimental effects of TGFβ can be negated by expressing a dominant negative TGFβ receptor II.

HER2-CAR T cells can also be genetically engineered to actively benefit from the inhibitory signals generated by the tumor environment, by converting inhibitory into stimulatory signals. Many tumors secrete IL4 to create a TH2-polarized environment, and expression of chimeric IL4 receptors consisting of the ectodomain of the IL4 receptor and the endodomain of the IL7Rα or the IL-2β chain enable T cells to proliferate in the presence of IL4 and retain their effector function including TH1-polarization. Chimeric TGFβ receptors are another example of these ‘signal converters’. For example, linking the extracellular domain of the TGFβ Receptor II endodomain of toll-like receptor (TLR) 4 results in a chimeric receptor that not only renders T cells resistant to TGFβ results in a chimeric receptor that not only renders T cells resistant to TGFβ.

Because enhancing the potency of HER2-CAR T cells may result in ‘on target/off cancer’ toxicity, additional genetic modifications to increase safety may be utilized, such as an inducible suicide gene (for example, caspase-9) or inhibitory receptors to limit the effector function of T cells to tumor sites. For example, an inhibitory receptor may comprise an extracellular domain that binds to molecules on normal tissue that is not present on the tumor, a transmembrane domain, and an intracellular domain that transmits a ‘negative signal’, such as one derived, for example, from PD-1.

In particular embodiments, the immune cells that comprise the HER2-specific CAR are also antigen-specific, such as antigen-specific T cells. Although the immune cell may be specific for any kind of antigen, in specific embodiments the antigen is a cancer antigen, a virus, or a bacteria. In cases wherein the immune cell is specific for a virus, for example, the virus may be of any kind, and in some cases a plurality of HER2-specific CAR immune cells that are antigen-specific for different viruses are provided to the individual. For example, in a plurality of HER2-specific CAR immune cells that are provided to the individual, one cell may be a HER2-specific CAR immune cell that is specific for CMV, whereas another HER2-specific CAR immune cell in the plurality may be specific for EBV. Some viruses to which a HER2-specific CAR immune cell is specific to include at least EBV, CMV, Adenovirus, BK, HHV6, RSV, Influenza, Parainfluenza, Bocavirus, Coronavirus, LCMV, Mumps, Measles, Metapneumovirus, Parvovirus B, Rotavirus, and West Nile Virus, for example.

### IV. Pharmaceutical Compositions

Provided herein are pharmaceutical compositions comprising the genetically engineered immune cells, e.g., genetically engineered HER2-specific CAR T cells.

In accordance with this disclosure, the term “pharmaceutical composition” relates to a composition for administration to an individual. In a preferred embodiment, the pharmaceutical composition comprises a composition for parenteral, transdermal, intraluminal, intra-arterial, intrathecal or intravenous administration or for direct injection into a cancer. It is in particular envisaged that said pharmaceutical composition is administered to the individual via infusion or injection. Administration of the suitable compositions may be effected by different ways, e.g., by intravenous, subcutaneous, intraperitoneal, intramuscular, topical or intradermal administration.

The pharmaceutical composition of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen may be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. An example of a dosage for administration might be in the range of 1×10⁶/m² to 1×10¹⁰/m². Particularly preferred dosages are recited herein below. Progress can be monitored by periodic assessment.

The CAR cell compositions of the disclosure may be administered locally or systemically. Administration will generally be parenteral, e.g., intravenous; DNA may also be administered directly to the target site, e.g., by biolistic delivery to an internal or external target site or by catheter to a site in an artery. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the pharmaceutical composition.

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR molecule as described herein and/or regulatory elements therefor.

In particular embodiments, there are one or more apparatuses in the kit suitable for extracting one or more samples from an individual. The apparatus may be a syringe, scalpel, and so forth.

In some cases, the kit, in addition to the cell therapy embodiments disclosed herein, also includes a second cancer therapy, such as chemotherapy, hormone therapy, and/or immunotherapy, for example. The kit(s) may be tailored to a particular cancer for an individual and comprise respective second cancer therapies for the individual.

### V. Therapeutic Uses of CARs and Host T-Cells Comprising CARs

In various embodiments CAR constructs, nucleic acid sequences, vectors, host cells, as contemplated herein and/or pharmaceutical compositions comprising the same are used for the prevention, treatment or amelioration of a cancerous disease, such as a tumorous disease, or any disease wherein vasculature is a detriment. In particular embodiments, the pharmaceutical composition of the present disclosure may be particularly useful in preventing, ameliorating and/or treating cancer, including cancer having solid tumors, for example.

In particular embodiments, provided herein is a method of treating an individual for cancer, comprising the step of providing a therapeutically effective amount of a plurality of any of cells of the disclosure to the individual. In certain aspects, the cancer is a solid tumor, and the tumor may be of any size, but in specific embodiments, the solid tumors are about 2 mm or greater in diameter. In certain aspects, the method further comprises the step of providing a therapeutically effective amount of an additional cancer therapy to the individual.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present invention contemplates, in part, cells, CAR constructs, nucleic acid molecules and vectors that can administered either alone or in any combination using standard vectors and/or gene delivery systems, and in at least some aspects, together with a pharmaceutically acceptable carrier or excipient. In certain embodiments, subsequent to administration, said nucleic acid molecules or vectors may be stably integrated into the genome of the subject.

In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject or individual in the need thereof an effective amount of immune cells, e.g., T cells or cytotoxic T lymphocytes, harboring a HER2 CAR; a nucleic acid sequence encoding a HER2 CAR; a vector comprising a nucleotide sequence encoding a HER2 CAR or both, as described herein and/or produced by a process as described herein.

Possible indications for administration of the composition(s) of the exemplary CAR cells are cancerous diseases, including tumorous diseases, including sarcoma, glioblastoma, breast, prostate, lung, and colon cancers or epithelial cancers/carcinomas such as breast cancer, colon cancer, prostate cancer, head and neck cancer, skin cancer, cancers of the genitourinary tract, e.g. ovarian cancer, endometrial cancer, cervical cancer and kidney cancer, lung cancer, gastric cancer, cancer of the small intestine, liver cancer, pancreatic cancer, gall bladder cancer, cancers of the bile duct, esophagus cancer, cancer of the salivary glands and cancer of the thyroid gland. The administration of the composition(s) of the disclosure is useful for all stages and types of cancer, including for minimal residual disease, early cancer, advanced cancer, and/or metastatic cancer and/or refractory cancer, for example, wherein the cancer is associated with pathogenic vascularization.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Particular doses for therapy may be determined using routine methods in the art. However, in specific embodiments, the T cells are delivered to an individual in need thereof once, although in some cases it is multiple times, including 2, 3, 4, 5, 6, or more times. When multiple doses are given, the span of time between doses may be of any suitable time, but in specific embodiments, it is weeks or months between the doses. The time between doses may vary in a single regimen. In particular embodiments, the time between doses is 2, 3, 4, 5, 6, 7, 8, 9, 10, or more weeks. In specific cases, it is between 4-8 or 6-8 weeks, for example. In specific embodiments, one dose includes at least 1×10⁴/m², 1×10⁵/m², 1×10⁶/m², 1×10⁷/m², 1×10⁸/m², 1×10⁹/m², or 1×10¹⁰/m². In particular embodiments, one dose includes no more than 1×10⁴/m², 1×10⁵/m², 1×10⁶/m², 1×10⁷/m², 1×10⁸/m², 1×10⁹/m², or 1×10¹⁰/m². In certain embodiments an individual is given a cell dose in the range of 1×10⁴/m² to 1×10¹⁰/m²; 1×10⁴/m² to 1×10⁹/m²; 1×10⁴/m² to 1×10⁸/m²; 1×10⁴/m² to 1×10⁷/m²; 1×10⁴/m² to 1×10⁶/m²; or 1×10⁴/m² to 1×10⁵/m²; 1×10⁵/m² to 1×10¹⁰/m²; 1×10⁵/m² to 1×10⁹/m²; 1×10⁵/m² to 1×10⁸/m²; 1×10⁵/m² to 1×10⁷/m²; 1×10⁵/m² to 1×10⁶/m²; 1×10⁶/m² to 1×10¹⁰/m²; 1×10⁶/m² to 1×10⁹/m²; 1×10⁶/m² to 1×10⁸/m²; 1×10⁶/m² to 1×10⁷/m²; 1×10⁷/m² to 1×10¹⁰/m²; 1×10⁷/m² to 1×10⁹/m²; 1×10⁷/m to 1×10⁸/m²; 1×10⁸/m² to 1×10¹⁰/m²; 1×10⁸/m² to 1×10⁹/m²; or 1×10⁹/m² to 1×10¹⁰/m².

Embodiments relate to a kit comprising cells as defined herein, a bispecific single chain antibody construct as defined herein, a nucleic acid sequence as defined herein, a vector as defined herein and/or a host as defined herein. It is also contemplated that the kit of this disclosure comprises a pharmaceutical composition as described herein above, either alone or in combination with further medicaments to be administered to an individual in need of medical treatment or intervention.

In particular embodiments, there are pharmaceutical compositions that comprise cells that express HER2-specific CARs. An effective amount of the cells are given to an individual in need thereof.

By way of illustration, cancer patients or patients susceptible to cancer or suspected of having cancer may be treated as follows. T-cells modified as described herein may be administered to the patient and retained for extended periods of time. The individual may receive one or more administrations of the cells. In some embodiments, the genetically engineered cells are encapsulated to inhibit immune recognition and placed at the site of the tumor.

In particular cases the individual is provided with therapeutic T-cells engineered to comprise a CAR specific for HER2. The cells may be delivered at the same time or at different times, wherein the CARs for HER2 and another antigen are in separate cells. The cells may be delivered in the same or separate formulations. The cells may be provided to the individual in separate delivery routes. The cells may be delivered by injection at a tumor site or intravenously or orally, for example. Routine delivery routes for such compositions are known in the art.

Expression vectors that encode the HER2 CARs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the HER2-specific CAR and optionally another gene, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

The exemplary T cells that have been engineered to include the HER2 CAR construct(s) are then grown in culture under selective conditions and cells that are selected as having the construct may then be expanded and further analyzed, using, for example; the polymerase chain reaction for determining the presence of the construct in the host cells. Once the engineered host cells have been identified, they may then be used as planned, e.g. expanded in culture or introduced into a host organism.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

In another aspect, provided herein is a method of treating an individual having a tumor cell, comprising administering to the individual a therapeutically effective amount of cells expressing at least HER2-specific CAR. In a related aspect, provided herein is a method of treating an individual having a tumor cell, comprising administering to the individual a therapeutically effective amount of cells expressing at least HER2-specific CAR. In a specific embodiment, said administering results in a measurable decrease in the growth of the tumor in the individual. In another specific embodiment, said administering results in a measurable decrease in the size of the tumor in the individual. In various embodiments, the size or growth rate of a tumor may be determinable by, e.g., direct imaging (e.g., CT scan, MRI, PET scan or the like), fluorescent imaging, tissue biopsy, and/or evaluation of relevant physiological markers (e.g., PSA levels for prostate cancer; HCG levels for choriocarcinoma, and the like). In specific embodiments of the invention, the individual has a high level of an antigen that is correlated to poor prognosis. In some embodiments, the individual is provided with an additional cancer therapy, such as surgery, radiation, chemotherapy, hormone therapy, immunotherapy, or a combination thereof.

In specific embodiments, one does not utilize lymphodepleting therapy of any kind prior to T-cell transfer, although in some embodiments one does utilize lymphodepleting therapy. Examples of lympodepleting therapy includes certain chemotherapy, radiation, chemotherapy plus radiation, or other means such as monoclonal antibodies.

In certain embodiments of methods of the disclosure, there is no delivery of one or more cytokines following exposure of the individuals to the cells of the disclosure although in alternative embodiments there is delivery of one or more cytokines to the individual post-infusion of the cells. Examples of cytokines include IL2, IL7, IL12, and IL15.

In certain embodiments, one can administer HER2-CAR T cells and one or more checkpoint antibodies (such as antibodies for CTLA4, PD-1, PD-L1, TIM3 or LAG3), thereby increasing T-cell activation and prolonging in vivo survival.

Embodiments relate to a kit comprising cells as defined herein, CAR constructs as defined herein, a nucleic acid sequence as defined herein, and/or a vector as defined herein. It is also contemplated that the kit of this disclosure comprises a pharmaceutical composition as described herein above, either alone or in combination with further medicaments to be administered to an individual in need of medical treatment or intervention.

### VI. Polynucleotide Encoding CARs

The present disclosure also encompasses a composition comprising a nucleic acid sequence encoding a CAR as defined herein and cells harboring the nucleic acid sequence. The nucleic acid molecule is a recombinant nucleic acid molecule, in particular aspects and may be synthetic. It may comprise DNA, RNA as well as PNA (peptide nucleic acid) and it may be a hybrid thereof.

It is evident to the person skilled in the art that one or more regulatory sequences may be added to the nucleic acid molecule comprised in the composition of the disclosure. For example, promoters, transcriptional enhancers and/or sequences that allow for induced expression of the polynucleotide of the disclosure may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62), or a dexamethasone-inducible gene expression system as described, e.g. by Crook (1989) EMBO J. 8, 513-519.

Furthermore, it is envisaged for further purposes that nucleic acid molecules may contain, for example, thioester bonds and/or nucleotide analogues. The modifications may be useful for the stabilization of the nucleic acid molecule against endo- and/or exonucleases in the cell. The nucleic acid molecules may be transcribed by an appropriate vector comprising a chimeric gene that allows for the transcription of said nucleic acid molecule in the cell. In this respect, it is also to be understood that such polynucleotides can be used for “gene targeting” or “gene therapeutic” approaches. In another embodiment the nucleic acid molecules are labeled. Methods for the detection of nucleic acids are well known in the art, e.g., Southern and Northern blotting, PCR or primer extension. This embodiment may be useful for screening methods for verifying successful introduction of the nucleic acid molecules described above during gene therapy approaches.

The nucleic acid molecule(s) may be a recombinantly produced chimeric nucleic acid molecule comprising any of the aforementioned nucleic acid molecules either alone or in combination. In specific aspects, the nucleic acid molecule is part of a vector.

The present disclosure therefore also relates to a composition comprising a vector comprising the nucleic acid molecule described in the present disclosure.

Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994). Alternatively, the polynucleotides and vectors of the disclosure can be reconstituted into liposomes for delivery to target cells. A cloning vector may be used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

In specific embodiments, there is a vector that comprises a nucleic acid sequence that is a regulatory sequence operably linked to the nucleic acid sequence encoding a CAR construct defined herein. Such regulatory sequences (control elements) are known to the artisan and may include a promoter, a splice cassette, translation initiation codon, translation and insertion site for introducing an insert into the vector. In specific embodiments, the nucleic acid molecule is operatively linked to said expression control sequences allowing expression in eukaryotic or prokaryotic cells.

It is envisaged that a vector is an expression vector comprising the nucleic acid molecule encoding a CAR construct defined herein. In specific aspects, the vector is a viral vector, such as a lentiviral vector. Lentiviral vectors are commercially available, including from Clontech (Mountain View, Calif.) or GeneCopoeia (Rockville, Md.), for example.

The term “regulatory sequence” refers to DNA sequences that are necessary to effect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the recited vector is an expression vector, in certain embodiments. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements that are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al. Cancer Immunol Immunother (2001) 50(3), 141-150) or pSPORT1 (GIBCO BRL).

In some embodiments, the expression control sequences are eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the disclosure may follow.

Additional regulatory elements may include transcriptional as well as translational enhancers. Advantageously, the above-described vectors of the disclosure comprises a selectable and/or scorable marker. Selectable marker genes useful for the selection of transformed cells are well known to those skilled in the art and comprise, for example, antimetabolite resistance as the basis of selection for dhfr, which confers resistance to methotrexate (Reiss, Plant Physiol. (Life-Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus that confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, P1. Sci. 116 (1996), 59-72; Scikantha, J. Bact. 178 (1996), 121), green fluorescent protein (Gerdes, FEBS Lett. 389 (1996), 44-47) or beta-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule can be used in a cell, alone, or as part of a vector to express the encoded polypeptide in cells. The nucleic acid molecules or vectors containing the DNA sequence(s) encoding any one of the CAR constructs described herein is introduced into the cells that in turn produce the polypeptide of interest. The recited nucleic acid molecules and vectors may be designed for direct introduction or for introduction via liposomes, or viral vectors (e.g., adenoviral, retroviral) into a cell. In certain embodiments, the cells are T-cells, CAR T-cells, NK cells, NKT-cells, MSCs, neuronal stem cells, or hematopoietic stem cells, for example.

In accordance with the above, the present disclosure relates to methods to derive vectors, particularly plasmids, cosmids, viruses and bacteriophages used conventionally in genetic engineering that comprise a nucleic acid molecule encoding the polypeptide sequence of a CAR defined herein. In certain cases, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the recited polynucleotides or vector into targeted cell populations. Methods that are well known to those skilled in the art can be used to construct recombinant vectors; see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the disclosure can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

### VII. Vectors Generally

The disclosure encompasses immune cells that are engineered to harbor a CAR-expressing DNA polynucleotide, which in certain embodiments is a vector having an expression construct or referred to as an expression vector. The elements of a vector may be routinely selected in the art, although those vectors for the present disclosure are unique in their incorporation of a HER2-specific CAR.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the beta-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

B. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with beta-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

C. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may be a viral vector that encodes one or more CARs of the invention. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

D. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

E. Vector Delivery and Cell Transformation

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

F. Ex Vivo Transformation

Methods for transfecting eukaryotic cells and tissues removed from an organism in an ex vivo setting are known to those of skill in the art. Thus, it is contemplated that cells or tissues may be removed and transfected ex vivo using nucleic acids of the present invention. In particular aspects, the transplanted cells or tissues may be placed into an organism. In preferred facets, a nucleic acid is expressed in the transplanted cells.

### VIII. Combination Therapy

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that cell therapy could be used similarly in conjunction with chemotherapeutic, radiotherapeutic, or immunotherapeutic intervention, in addition to other pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, such as wherein cells of the present disclosure are “A” and the secondary agent, such as radiotherapy, immunotherapy, or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Combination anti-cancer agents include, for example, acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estrarnustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone: didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin: neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer, or any analog or derivative variant of the foregoing and also combinations thereof.

In specific embodiments, chemotherapy for the individual is employed in conjunction with the invention, for example before, during and/or after administration of the invention.

B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention.

In certain embodiments, the immunotherapy is an antibody against HER2, such as trastuzumab (marketed as Herceptin®), 800E6, F5cys, or Pertuzumab.

D. Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A Phase I Clinical Trial of Autologous HER2 CMV Bispecific Chimeric Antigen Receptor T Cells for the Adoptive Immunotherapy of Glioblastoma

The outcome for patients with Glioblastoma (GBM) remains poor. T-cell therapy holds the promise to improve outcomes for GBM patients since it does not rely on the cytotoxic mechanisms of conventional therapies. It has been shown in preclinical studies that HER2 and the CMV-derived protein pp65 (CMVpp65) are T-cell therapy targets for GBM. Based on these findings a Phase I/II clinical study (NCT01109095) was developed with CMVpp65-specific T cells expressing a HER2-specific chimeric antigen receptor (CAR) with a CD28.ζ signaling domain (HER2-CAR.CMV-T cells). The phase I/II clinical study was developed to determine the safety, persistence, and anti-GBM effects of escalating doses (1×10⁶/m² to 1×10⁸/m²) of autologous HER2-CAR.CMV-T cells in patients with recurrent/refractory HER2+ GBM. Sixteen CMV-seropositive patients with HER2-positive GBM aged 11-70 years (median 49 years) were enrolled. HER2-CAR.CMV-T cells were successfully generated from all patients. T-cell products contained HER2-CAR expressing T cells as judged by FACS analysis (median: 67% (range: 46-82) %), and pp65CMV-specific T cells as judged by IFN-γ Elispot assays (median 985.5 (range 390 to 1292) SFC/10⁵ T cells). Infusions of 1×10⁶/m², 3×10⁶/m², 1×10⁷/m², 3×10⁷/m² or 1×10⁸/m² HER2-CAR.CMV-T cells were well tolerated without systemic side effects and no dose limiting toxicity was observed. HER2-CAR.CMV-T cells were detected for up to 10 weeks post infusion as judged by real-time PCR. Out of fifteen evaluable patients 10 had progressive disease, 1 had a partial response with a ˜62% reduction in tumor volume lasting 8 months, 1 patient had stable disease lasting 4 months and 3 patients have stable disease and are currently alive with a follow up of 16 to >22 months, after T cell infusion. This first evaluation of the safety and efficacy of autologous HER2.CMV-T cells in GBM patients shows that cells could persist for 10 weeks without evident toxicities. Clinical benefit was observed in 33% of patients setting the stage for studies that combine HER2-CAR.CMV-T cells with other immunomodulatory approaches to enhance their expansion and anti-GBM activity.

### Example 2

HER2-Specific Chimeric Antigen Receptor-Modified T Cells for the Immunotherapy of HER2-Positive Sarcoma

The present Example concerns the use of HER2-specific CAR T cells for sarcoma.

Patient Characteristics

The clinical and disease-specific characteristics of the 19 exemplary patients, who received HER2-CAR T cells are summarized in Table 1.

TABLE 1
Patient characteristics

| | | | Prior Treatment | | | |
|---|---|---|---|---|---|---|
| | Age (y)/ | Dx | | | | Other and/or Investigational |
| P | sex | (Stage) | Chemotherapy | Surgery | XRT | Agents |
| 1 | 21.2/F | OS | (1) MAPIE; (2) Ifos, | LS | Y | (1) Avastin; (2) Sunib |
| | | (M) | VP16, HDMTX | | | |
| 2 | 17.4/F | OS | (1) MAP; (2) Ifos | LS; M(3) | N | (1) L-MTP-PE |
| | | (L) | | | | |
| 3 | 14.0/F | OS | (1) MAP; (2) Ifos, VP16, | LS; M(5) | Y | (1) GCB; (2) L-MTP-PE, Oral |
| | | (M) | HDMTX | | | CPM; (3) Sunib |
| 4 | 17.1/M | OS | (1) MAPIE | LS; M(4) | Y | (1) SCH717454 (2) L-MTP-PE; |
| | | (L) | | | | (3) Sunib; (4) GCB, Docetaxel, |
| | | | | | | Avastin (5) Doxil |
| 5 | 7.7/F | OS | (1) MAP; (2) Ifos, Carbo, | LS; M(3) | N | (1) L-MTP-PE, GCB; (2) L- |
| | | (M) | VP16 | | | MTP-PE, oral CPM; (3) |
| | | | (3) HD Ifos, HDMTX; (4) | | | Denosumab, Doxil |
| | | | HDMTX | | | |
| 6 | 25.3/F | OS | (1) MAP; (2) Doxo, Ifos | Primary | N | (1) L-MTP-PE, Docetaxel |
| | | (L) | (3) HDMTX; (4) Ifos | en bloc; | | (2) GCB, Avastin, L-MTP-PE |
| | | | | M(1) | | |
| 7 | 29.6/M | OS | (1) MAPIE | LS; M(3) | N | (1) L-MTP-PE (2) L-MTP-PE, |
| | | (L) | | | | GCB |
| 8 | 15.4/F | OS | (1) MAP; (2) HD Ifos; (3) | Amp-for | Y | (1) GCB; (2) Doxil, Avastin; (3) |
| | | (L) | HDMTX, Cis; | quarter; | | L-MTP-PE; (4) Sorafenib |
| | | | (4) Oral CPM | M(4) | | |
| 9 | 21.1/F | OS | (1) MAP; (2) HD Ifos | LS; M(2) | Y | (1) Doxil; (2) L-MTP-PE |
| | | (M) | | | | |
| 10 | 14.0/F | PNET | (1) VDCIE; (2) Carbo, | Rt | Y | (1) Sorafenib |
| | | (L) | VP16, Mel + auto; (3) | kidney; | | |
| | | | Metronomic VCR; (4) | M(1) | | |
| | | | TMZ, irinotecan | | | |
| 11 | 16.6/M | OS | (1) MAP; (2) Ifos, VP16 | LS; M(3) | N | (1) L-MTP-PE; (2) Sunib; (3) |
| | | (L) | | | | Doxil |
| 12 | 11.3/M | DSCRT | (1) VDC x2; (2) | Primary | Y | (1) Sorafenib; (2) PEG-IFN |
| | | (L) | Topotecan, CPM | en bloc; | | |
| | | | (3) TMZ, Irinotecan; (4) | Hep emb | | |
| | | | Vinorelbine, CPM | | | |
| 13 | 20.6/M | OS | (1) MAPIE; (2) TMZ | LS; M(4) | Y | (1) L-MTP-PE; (2) Avastin, |
| | | (M) | | | | GCB, Docetaxel; (3) Sorafenib |
| 14 | 21.8/F | OS | (1) Intra-arterial Cis, | LS; M(3) | N | (1) L-MTP-PE, oral CPM |
| | | (L) | Doxo, HDMTX, Ifos; (2) | | | (2) Doxil, Avastin, HDMTX |
| | | | Intra-pleural Cis x 2; (3) | | | |
| | | | HDMTX | | | |
| 15 | 11.0/M | OS | (1) MAP | Amp; M(6) | Y | (1) L-MTP-PE; (2) GCB; (3) |
| | | (M) | | | | Doxil, Avastin; (4) Sorafenib; |
| | | | | | | (5) Pazib, Lapib |
| 16 | 19.3/M | OS | (1) Doxo, intra-arterial | LS; M(2) | N | (1) L-MTP-PE; (2) SCH717454 |
| | | (L) | Cis, HD MTX | | | (IGF-IR MAb); (3) IFN; (4) |
| | | | (2) HD Ifos; (3) oral CPM; | | | Sunib d |
| | | | (4) Doxo, Ifos | | | |
| 17 | 16.8/M | ES | (1) VDC-IE; (2) VCR, | M(1) | Y | (1) Temsirolimus, IMC A12 (2) |
| | | (M) | TMZ, irinotecan; | | | Doxil; |
| | | | (3) vinoralbine, CPM | | | (3) Vori, Pazib; (4) Pazib, Lapib |
| 18 | 14.5/F | OS | (1) MAPIE | LS; M(3) | N | none |
| | | (L) | | | | |
| 19 | 16.5/M | OS | (1) MAP; (2) HD Ifos, | LS; M(5) | Y | (1) Imetelstat |
| | | (M) | VP16 x 2 | | | |

DSRCT: Desmoplastic small round cell tumor
ES: Ewing's sarcoma
OS: Osteosarcoma
PNET: Primitive neuroectodermal tumor
M: Metastatic
L: Localized
Auto: Autologous transplant
Carbo: Carboplatin
Cis: Cisplatin
CPM: Cyclophosphamide
Doxo: Doxorubicin
GCB: Gemcitabine
HD: High dose
IE: Ifos, VP16
Ifos: Ifosfamide
MAP: MTX, Doxo, Cis
Mel: Melphalan
MTX: Methotrexate
TMZ: Temozolomide
VCR: Vincristine
VDC: VCR, Doxo, CPM
VP16: Etoposide
Amp: Amputation
LS: Limb salvage
Hep emb: Hepatic embolization
M: Metastatectomy
(#): number of procedures
N: No
Y: Yes
Lapib: Lapatanib
Pazib: Pazaponib
Sunib: Sunitinib
Vori: Vorinostat

Their median age at the time of T-cell infusion was 17 years (range: 7.7-29.6). Sixteen patients had osteosarcoma, 1 had Ewing's sarcoma, 1 a primitive neuroectodermal tumor (PNET), and 1 had a desmoplastic small round cell tumor (DSRCT). HER2 positivity was confirmed by immunohistochemistry (Table 2). All patients had refractory/recurrent metastatic disease at the time of T-cell infusion, and had failed one or more conventional chemotherapy regimens. Seventeen of 19 had undergone metastatectomies (median 4; range: 1 to 6), 11/19 patients had received radiation therapy, and 18 of 19 patients had received one or more salvage regimens (median: 3; range: 1 to 5) prior to T-cell infusion. All enrolled patients had performance status of ≧60 (Karnofsky/Lansky scale), and a normal left ventricular ejection fraction (LVEF).

Generation and Characterization of HER2-CAR T Cells

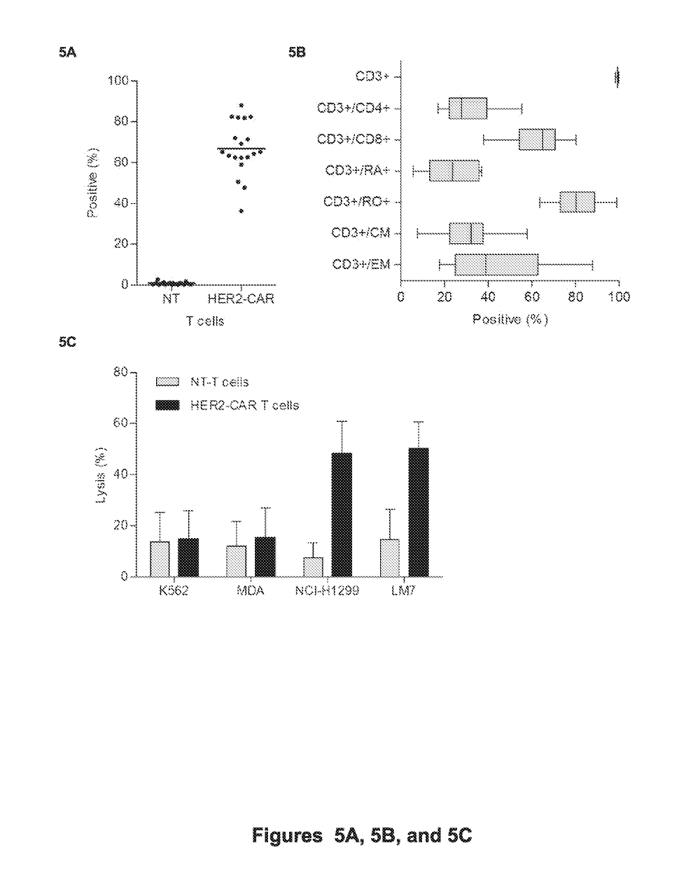

HER2-CAR T cells were successfully generated for all patients. The median time to manufacture the cell product for clinical use was 13.5 days (range: 10 to 21). Greater than 97.8% of the transduced cells were CD3+ (mean: 99.2%, range: 97.8-99.6%), and both CD3+/CD8+ (mean: 62.7%, range: 37.8-80.1%) and CD3+/CD4+ (mean: 31.5%, range: 17.2-55.3%) subsets were present in all products (FIG. 5A). CAR T-cell products also contained naïve (CD3+/CD45RA+; mean: 22.6%; range 6.0-37.2%), effector memory (CD3+/CD45RO+/CCR7−/CD62L−; mean: 32.2%; range: 7.7-57.9%), and central memory T cells (CD3+/CD45RO+/CD62L+; mean: 45.8%; range: 17.9-88.0%). A median of 65.2% (range: 36.2-88%) of T cells were positive for HER2-CAR expression as judged by FACS analysis (FIG. 5B). In a standard ⁵¹chromium (Cr)-release cytotoxicity assay, HER2-CAR T cells had significant cytotoxic activity against HER2-positive (NCI-H1299, LM7) target cells, whereas non-transduced (NT)-T cells did not (p<0.0001; FIG. 5C). Only background killing was present when HER2-negative (K562, MDA-MB468) target cells were cultured with HER2-CAR and NT-T cells.

Administration and Safety of HER2-CAR T Cells

Patients received between 1×10⁴/m² to 1×10⁸/m² HER2-CAR T cells on 8 dose levels. Thirteen patients received 1 infusion; 4 patients 2, 1 patient 4 and 1 patient 9 infusions. None of the patients had adverse events related to the T-cell infusion except for one patient (P16) on the highest dose levels, who developed fever within 12 hours post T-cell infusion, which resolved with ibuprofen.

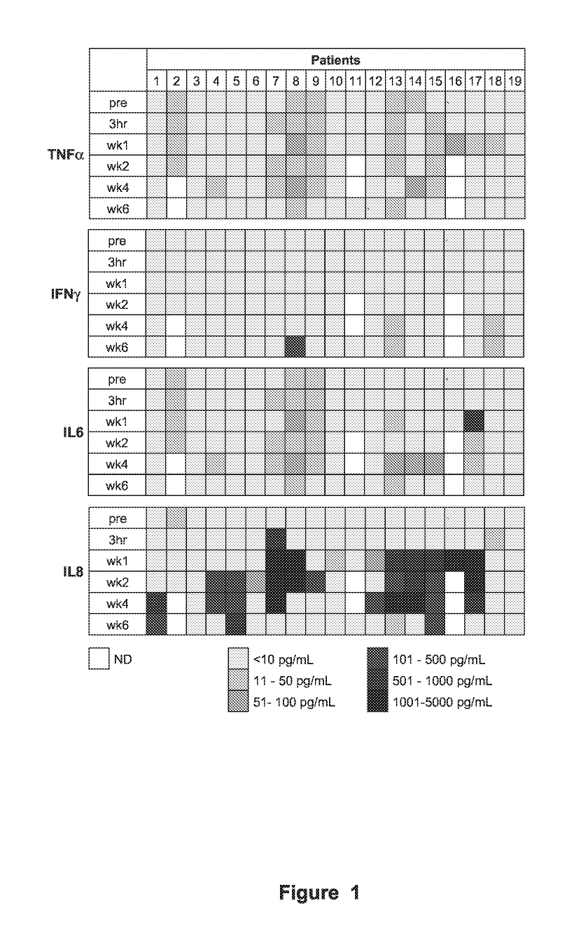

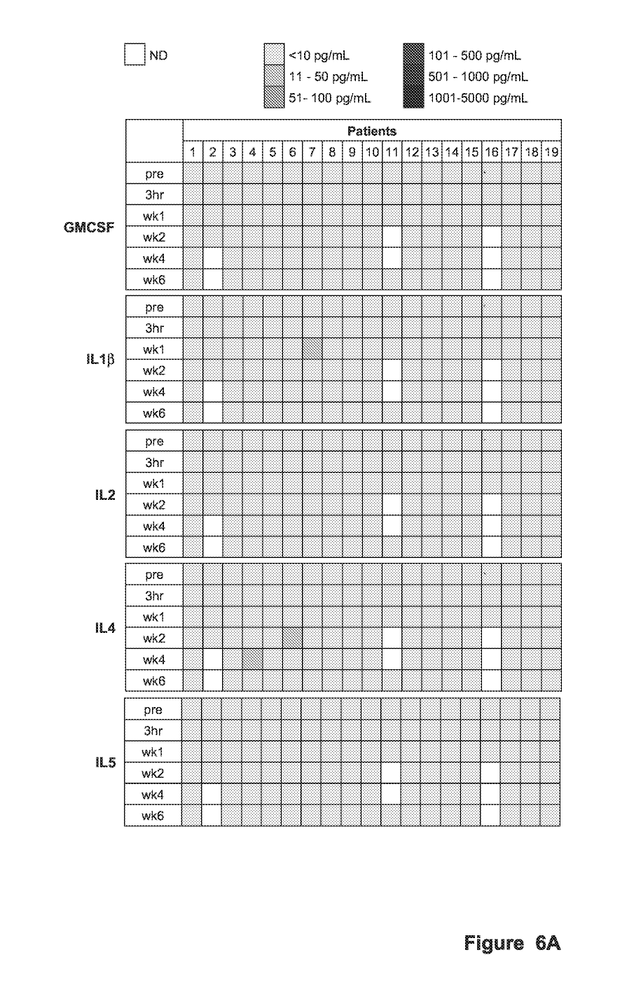

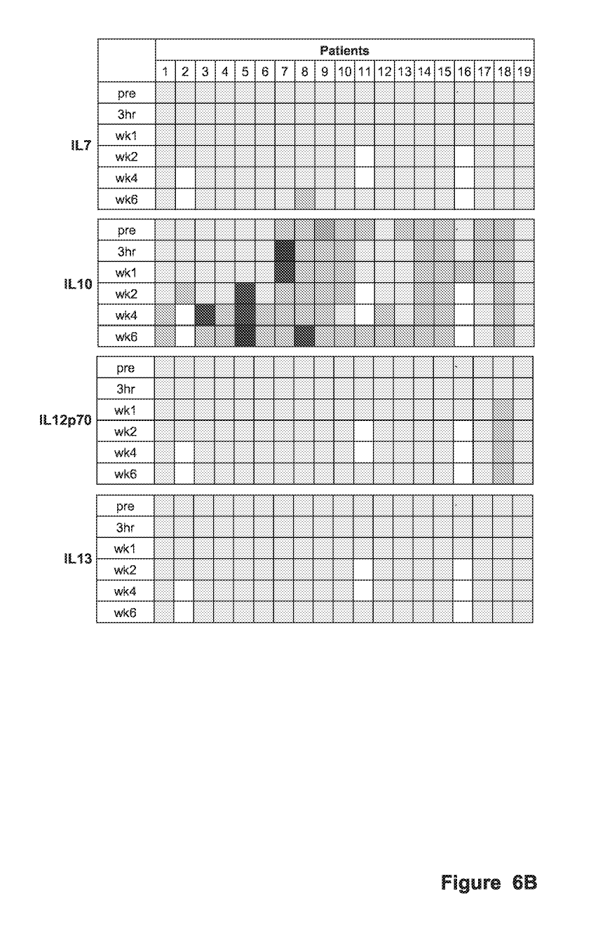

Concentrations of plasma cytokines (GM-CSF, IFNγ, IL1β, IL2, IL4, IL5, IL6, IL7, IL8, IL10, IL12p70, IL13, and TNF) were determined post infusion by multiplex analysis at 3 hours and again at 1, 2, 4, and 6 weeks post infusion (FIG. 1; FIG. 6). There was a significant increase (p<0.05) in the plasma concentration of IL8 as early as 1 week post infusion, and this persisted for up to 4 weeks. No significant change in any other cytokine was observed. At 6 weeks post infusion, repeat cardiac function studies showed LVEFs unchanged from baseline.

TABLE 2
Results of HER2 Immunohistochemistry

| P | Intensity Score | Grade |
|---|---|---|
| 1 | ++ | 1 |
| 2 | +++ | 4 |
| 3 | + | 1 |
| 4 | +++ | 3 |
| 5 | +++ | 4 |
| 6 | +++ | 3 |
| 7 | ++ | 3 |
| 8 | +++ | 4 |
| 9 | ++ | 3 |
| 10 | +++ | 4 |
| 11 | + | 2 |
| 12 | +++ | 4 |
| 13 | ++ | 2 |
| 14 | +++ | 4 |
| 15 | ++ | 2 |
| 16 | + | 1 |
| 17 | ++ | 2 |
| 18 | ++ | 3 |
| 19 | + | 1 |

Concentrations of plasma cytokines (GM-CSF, IFNγ, IL1β, IL2, IL4, IL5, IL6, IL7, IL8, IL10, IL12p70, IL13, and TNFα) were determined post infusion by multiplex analysis at 3 hours and again at 1, 2, 4, and 6 weeks post infusion (FIG. 1; FIG. 6). There was a significant increase (p<0.05) in the plasma concentration of IL8 as early as 1 week post infusion, and this persisted for up to 4 weeks. No significant change in any other cytokine was observed. At 6 weeks post infusion, repeat cardiac function studies showed LVEFs unchanged from baseline.

In Vivo Detection and Persistence of HER2-CAR T Cells

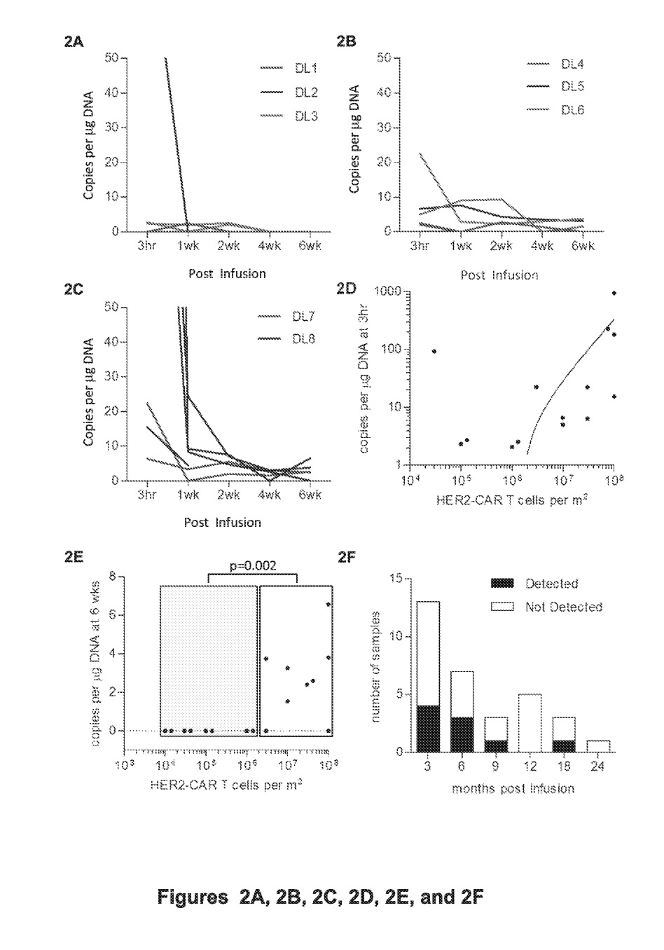

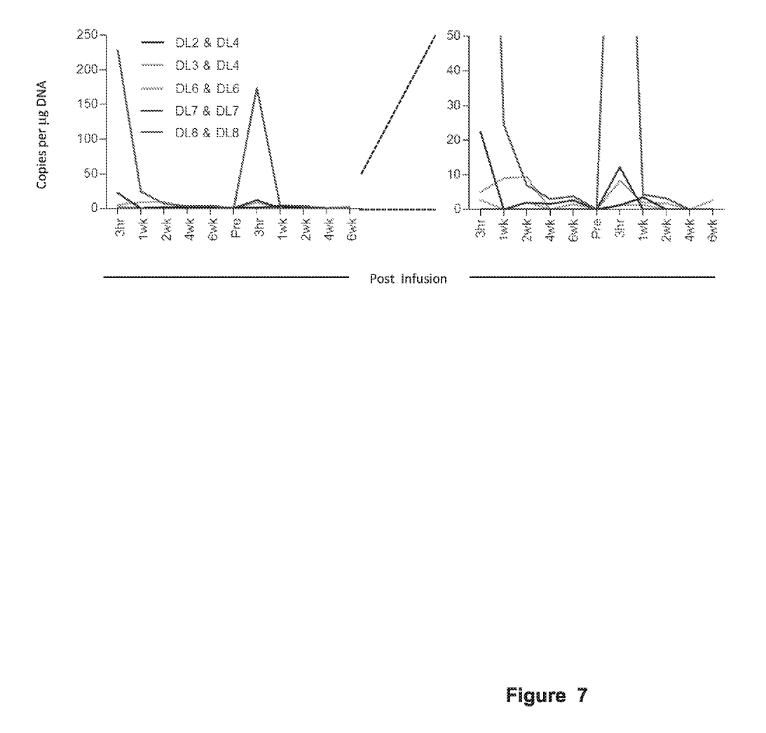

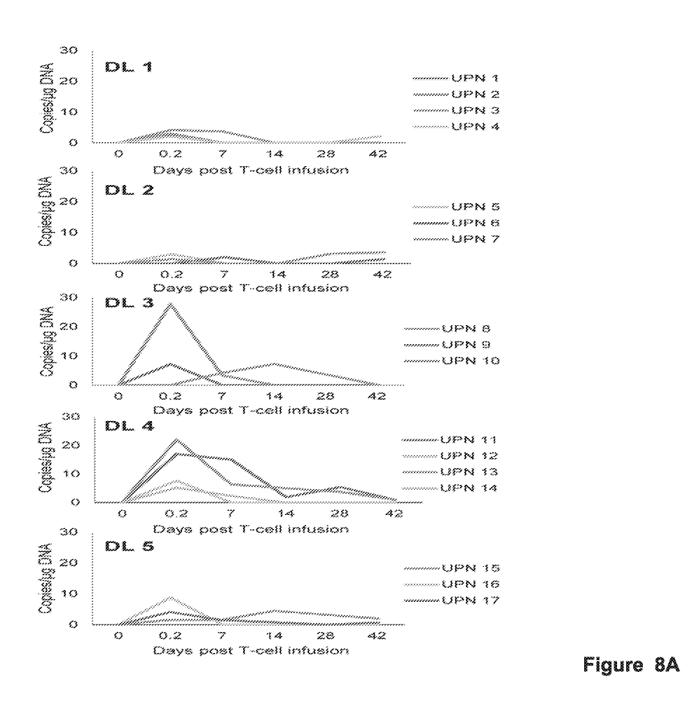

HER2-CAR T cells were detected in vivo by qPCR analysis of PBMC. From dose level 3 (1×10⁵/m²) and higher HER2-CAR T cells were detected in the peripheral blood of 14/16 patients (median: 6.5 copies per g DNA; range 0-944) (FIGS. 2A-C), and the copy number correlated with the infused T-cell dose (FIG. 2D). After the 3 hour time point, there was a rapid decline in the frequency of HER2-CAR T cells but low level signal could be detected 6 weeks post infusion in 7 of the 9 evaluable patients who had received greater than 1×10⁶/m² HER2-CAR T cells (≦1×10⁶/m² vs >1×10⁶/m²: p=0.002) (FIG. 2E). At 3 months we could detect HER2-CAR T cells in 4/13 evaluable subjects, at 6 months in 3/7, at 9 months in ½, at 12 months in 0/5, at 18 months in ½, and at 24 months in 0/1 patients (FIG. 2F). Thus, no evidence was detected for HER2-CAR T expansion in peripheral blood post infusion, these cells could persist long-term. Five patients received at least two doses of HER2-CAR T cells and there was a similar pattern of HER2-CAR T-cell persistence after both infusions (FIG. 7).

HER2-CAR T Cells Traffic to Tumor Sites

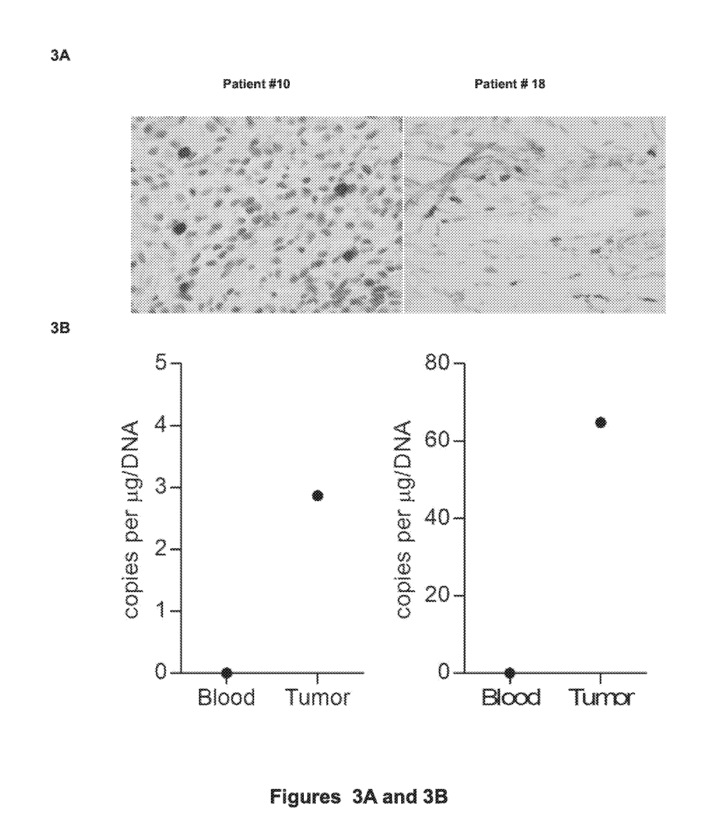

Five patients had tumors removed 9 to 15 weeks post HER2-CAR T-cell infusion. For 2/5 patients (P10, soft tissue metastasis; P18, metastatic lesion left femur) we received formalin-fixed slides and fresh frozen tissue. In both tumors, CD3-positive T cells were present by immunohistochemistry (FIG. 3A) and HER2-CAR T cells were present on qPCR analysis (FIG. 3B) even though no qPCR signal from HER2-CAR T cells was detected in the peripheral blood obtained at the same time as the resected tumor (FIG. 3B), indicating that HER2-CAR T cells either preferentially home to, persist or expand at, tumor sites. T cells were detected within the other three tumor samples by using a CD3-specific antibody, but lacked sufficient material for qPCR analysis.

Clinical Responses after HER2-CAR T-Cell Infusion

Clinical responses were measured by pre- and post T-cell infusion imaging as detailed elsewhere herein. These data are summarized in Table 3.

TABLE 3
Patient outcome

| | | Disease at T- | T-cell | | OS |
|---|---|---|---|---|---|
| P | Dx | cell infusion | dose | Outcome | (days) |
| 1 | OS | Right hip, | 1 × 10⁴/m² | PD | 1109* |
| | | Multiple | | | |
| | | bones | | | |
| 2 | OS | Sacrum | 1 × 10⁴/m² | NE | 34 |
| 3 | OS | Right hip | 1 × 10⁴/m² | PD | 584 |
| | | Lung | | | |
| 4 | OS | Lung | 3 × 10⁴/m² | PD; surgery/salvage chemotherapy for PD; | 874 |
| | | | | 2^{nd} infusion (1 × 10⁵/m²); PR for 9 months | |
| 5 | OS | Lung | 3 × 10⁴/m² | PD; 2^{nd} infusion (1 × 10⁵/m²); PD | 310 |
| 6 | OS | Lung | 1 × 10⁵/m² | PD | 107 |
| 7 | OS | Lung | 1 × 10⁵/m² | PD; 2^{nd} infusion (1 × 10⁵/m²); PD | 303 |
| 8 | OS | Pelvis, spine, | 1 × 10⁶/m² | PD | 120 |
| | | Lung | | | |
| 9 | OS | Lung | 1 × 10⁶/m² | PD | 151 |
| 10 | PNET | Sacrum | 3 × 10⁶/m² | PD; tumor removed (no necrosis) | 451 |
| 11 | OS | Lung/Pleura | 3 × 10⁶/m² | SD for 15 wks; tumor removed (no | 584* |
| | | | | necrosis); remains in remission | |
| 12 | DSCRT | Liver | 1 × 10⁷/m² | 8 additional infusions; SD for 14 months | 528* |
| 13 | OS | Lung | 1 × 10⁷/m² | PD | 268 |
| 14 | OS | Lung/Pleura | 3 × 10⁷/m² | 2^{nd} infusion; SD for 12 weeks; tumor | 446* |
| | | | | removed (≥90% necrosis); 2 additional | |
| | | | | infusions; remains in remission | |
| 15 | OS | Bone, chest | 3 × 10⁷/m² | PD | 156 |
| | | wall, brain, | | | |
| | | spine, | | | |
| | | marrow | | | |
| 16 | OS | Pleura, liver, | 1 × 10⁸/m² | NE | 389* |
| | | sub- | | | |
| | | diaphragmatic | | | |
| 17 | ES | Lung | 1 × 10⁸/m² | PD | 153 |
| 18 | OS | Left femur | 1 × 10⁸/m² | 2^{nd} infusion; SD for 12 wks; tumor | 290* |
| | | | | removed (no necrosis); remains in | |
| | | | | remission | |
| 19 | OS | Lung and | 1 × 10⁸/m² | PD | 164 |
| | | bone | | | |

P: Patient;
Dx: Diagnosis;
R( ): recurrence number;
PD: progressive disease;
SD: stable disease;
NE: not evaluable;
*alive

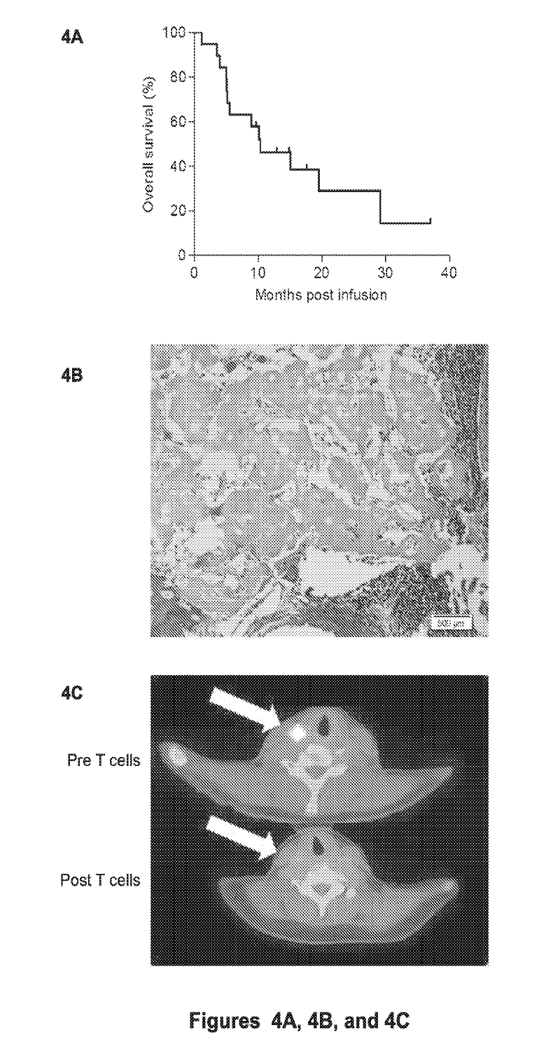

Of 17 evaluable patients, four had stable disease for 12 weeks to 14 months. One of the patients with progressive disease (P4) received salvage chemotherapy followed by a 2nd dose of T cells for metastatic lymph node disease. Following this second infusion, he had a partial response that lasted for 9 months (FIG. 4C). Three patients with stable disease (P11, P14, P18) received no additional therapy and had their residual tumor removed. The sample from P14 showed ≧90% necrosis, demonstrating antitumor activity of infused HER2-CAR T cells (FIG. 4B). All three of these patients remain in remission at 6, 12, and 16 months with no further treatment. With a median follow up of 10.1 months (range: 1.1 to 37 months) the median overall survival (OS) was 10.3 months (range: 5.1 to 29.1 months) (FIG. 4A).

Exemplary Materials and Methods

Subjects

This study (NCT00902044) was approved by the Institutional Review Board at Baylor College of Medicine and by the Food and Drug Administration. Patients were eligible for the study if they had a diagnosis of refractory or metastatic HER2-positive osteosarcoma (later modified to sarcoma) not treatable by surgical resection and with disease progression after receiving at least one prior systemic therapy. HER2-positivity was determined by immunohistochemistry. Patients had to have completed (and recovered from) experimental or cytotoxic therapies at least 4 weeks prior to study entry. Patients were excluded if they had abnormal left ventricular function (LVEF). In addition, patients with a serum bilirubin of >3× upper limit normal, ALT or AST >5× upper limit of normal, Hgb<9 g/dl, WBC<2,000/μl, ANC<1,000/μl, platelets <100,000/μl, were excluded as were patients with a Karnofsky/Lansky score of <50 or positive serology for human immunodeficiency virus.

Study Description

All patients had imaging with computer tomography (CT), magnetic resonance imaging (MRI), and/or positron emission tomography (PET) to assess overall disease burden prior to T-cell infusion. Patients received escalating doses of HER2-CAR T cells (1×10⁴-1×10⁸/m²) on 8 dose levels (DL); DL1: 1×10⁴/m², DL2: 3×10⁴/m², DL3: 1×10⁵/m², DL4: 1×10⁶/m², DL5: 3×10⁶/m², DL6: 1×10⁷/m², DL7: 3×10⁷/m², DL8: 1×10⁸/m². Peripheral blood samples were obtained prior to T-cell infusion and at pre-determined time points after infusion to evaluate for toxicity and T-cell persistence and expansion. Clinical response to HER2-CAR T cells was assessed by radiographic imaging 6 weeks after the T-cell infusion. Patients were eligible to receive additional T-cell infusions if they had clinical benefit, defined as a complete response, partial response, or stable disease. All patients were infused between June 2010 and March 2013. Follow up continued until Sep. 1, 2013.

Generation and Transduction of HER2-CAR T Cells

HER2-CAR T cells were generated according to current Good Manufacturing Practice (cGMP) guidelines. Briefly, peripheral blood mononuclear cells (PBMCs) were activated with immobilized CD3 antibody (Ortho Biotech) or immobilized CD3 and CD28 antibodies (P 6, 12, 13, 16, 17, 18; Miltenyi) and recombinant IL2 (100 U/ml; Proleukin, Chiron), and then transduced on day 3 with retroviral particles encoding a HER2.CD28. ζ-CAR in 24 well plates precoated with Retronectin (FN CH-296; Takara). After transduction, T cell lines were expanded in the presence of IL2 (50-100 U/ml) added twice weekly until the specified cell dose was achieved. After expansion, HER2-CAR T cells were tested for sterility, HLA-identity, immunophenotype, and HER2-specificity at the time of cryopreservation. Specificity was tested in a 4-hour 51Cr-release cytotoxicity assay.

Clinical Response Criteria

Clinical response to T-cell infusion was evaluated by comparing disease identified by CT, MRI, and/or PET imaging obtained pre-infusion to images obtained 6 weeks post infusion or as clinically indicated. Re-biopsy of residual masses was not mandatory for study participation. All responses were determined using RECIST.

Statistical Analysis

The Phase I/II trial utilized the modified continual reassessment method (mCRM) in order to determine the maximum tolerated dose. Three patients were enrolled on dose level 1, 2 patients on dose level 2 through 7, and 4 patients on dose level 8. Transgene expression and multiplex analyses were summarized over time using descriptive statistics. The significance between groups was determined by t-test or using the Fisher's exact test. A p-value less than 0.05 was considered statistically significant. The survival curve was constructed using the Kaplan Meier method.

HER2 Immunohistochemistry

HER2 was detected by phospho-HER2 immunohistochemistry as previously described. HER2 staining was scored by an independent pathologist for % positive cells (Grade 1 (1-25%), Grade 2 (26-50%), Grade 3 (51-75%), Grade 4 (76-100%)), and intensity (0, +, ++, +++). For patients to be considered HER2-positive, tumors had to have 1% to 25% positive cells (Grade 1) and an intensity score of ‘+’.

Generation of Retroviral Construct

The generation of the HER2-CAR has been previously described. Briefly, the HER2-specific murine scFv FRP5 was cloned into a SFG retroviral vector containing a short hinge, a CD28 transmembrane domain, and a CD28ζ signaling domain. A clinical grade packaging cell line was generated using PG13 cells (gibbon ape leukemia virus pseudotyping packaging cell line; CRL-10686, ATCC) as previously described. The highest-titer clone was used to establish a master cell bank, which was used to produce a clinical batch of virus.

Flow Cytometry

A FACSCalibur instrument (Becton Dickinson, San Jose, Calif.) and CellQuest software (Becton Dickinson) was used for flow cytometric analysis. Monoclonal antibodies (MAbs) were obtained from Becton Dickinson and included anti-CD3, -CD4, -CD8, -CD16, -CD19, -CD56, -CD62L, -CCR7, -TCRα/β, and -TCRγ/δ. HER2-CAR expression was detected with a murine scFV-specific MAb (Jackson ImmunoResearch Laboratories). Negative controls included isotype antibodies.

Multiplex Analysis

A 13-plex human cytokine/chemokine bead array assay kit (Millipore) was used to measure; GM-CSF, IFNγ, IL1β, IL2, IL4, IL5, IL6, IL7, IL8, IL10, IL12p70, IL13, and TNFα. Each undiluted plasma sample was assayed in duplicate according to the protocol provided by the manufacturer.

Real-Time PCR Assay

A FRP5-specific primer and TaqMan probe (Applied Biosystems) were used to detect HER2-CAR T cells. DNA was extracted with the QIAamp DNA Blood Mini Kit (Qiagen) and qPCR was performed in triplicates using the ABI RPISM 7900HT Sequence Detection System (Applied Biosystems). The baseline range was set at cycles 6-15, with the threshold at 10 SDs above the baseline fluorescence. To generate DNA standards, we established serial dilution of DNA plasmids encoding each specific cassette.

### Example 3

HER2 Chimeric Antigen Receptor Modified CMV-Specific T-Cells for Progressive Glioblastoma: A Phase I Dose-Escalation Trial

The present example extends the subject matter of Example 1 concerning HER2-specific CARs for glioblastoma treatment.

This example describes a 2nd generation HER2-CAR with a CD28.ζ endodomain and an initial safety evaluation of up to 1×10⁸/m² HER2-CAR T cells in patients with sarcoma demonstrated no evident toxicity; however, T-cell persistence was limited.¹⁹ In specific embodiments, one can increase the expansion and persistence of adoptively transferred T-cells by relying on the expression of CARs in T-cells with defined antigen specificity, including T-cells specific for human herpes viruses. These cells not only provide antitumor activity through their CAR, but also receive appropriate co-stimulation following native T-cell receptor (αβPTCR) engagement by human herpes virus latent-antigens presented by professional antigen-presenting cells.¹¹ Since human herpes virus 5 (cytomegalovirus, CMV) is present both in latently infected leukocytes and in subsets of GBMs,²⁰⁻²³ there was developed a Phase 1 dose-escalation study of autologous T cells specific for the CMV antigen pp65 that were genetically modified to express a HER2-CAR with a CD28.ζ signaling domain (HER2/CMV T-cells). This example demonstrates the safety, persistence and anti-tumor activity of the infused cells in patients with recurrent/progressive GBM.

Introduction

Glioblastoma (GBM) is the most aggressive primary brain cancer. 1,2 Despite multimodal therapy that combines maximal surgical resection with post-operative adjuvant chemo-radiotherapy the 5-year overall survival (OS) rates have remained poor, <4% for adults and ˜16% for children.^{1,2} Tumor-targeted immunotherapy has the potential to improve outcomes because it does not rely on the cytotoxic mechanisms of conventional therapies to which GBM cells are resistant. Indeed, results from completed early phase clinical trials with peptide, tumor cell, or dendritic cell (DC) vaccines in GBM patients have been encouraging, demonstrating clinical benefit.³⁻⁵

Among other forms of immunotherapies, the adoptive transfer of chimeric antigen receptor (CAR)⁶ modified T-cells has shown significant antitumor activity in clinical studies for the treatment of CD19-positive hematological malignancies.⁷⁻⁹ However, the clinical experience for solid tumors and brain tumors is limited.¹⁰⁻¹³ CARs recognize antigens expressed on the cell surface of cancer cells, and for GBM-directed CAR T-cell therapy several antigens are actively being studied in preclinical models including IL13Rα2, EphA2, EGFRvIII, and HER2¹⁴⁻¹⁷ For example, HER2-CAR T cells kill both “bulk” glioma cells and gliomainitiating cells and have potent antitumor activity in preclinical GBM xenograft models.¹⁴

The inventors developed a 2nd generation HER2-CAR with a CD28.ζ endodomain and the initial safety evaluation of up to 1×10⁸/m² HER2-CAR T cells in patients with sarcoma demonstrated no evident toxicity; however, T-cell persistence was limited.¹⁹ One strategy to increase the expansion and persistence of adoptively transferred T-cells relies on the expression of CARs in T-cells with defined antigen specificity, including T-cells specific for human herpesviruses, for example.¹¹ These cells not only provide antitumor activity through their CAR, but also receive appropriate co-stimulation following native T-cell receptor (αβTCR) engagement by human herpesvirus latent-antigens presented by professional antigen-presenting cells.¹¹ Since human herpes virus (cytomegalovirus, CMV) is present both in latently infected leukocytes and in subsets of GBMs,²⁰⁻²³ a Phase 1 dose-escalation study was developed of autologous T cells specific for the CMV antigen pp65 that were genetically modified to express a HER2-CAR with a CD28.ζ signaling domain (HER2/CMV T-cells). This example demonstrates the safety, persistence and anti-tumor activity of the infused cells in patients with recurrent/progressive GBM.

Methods

Study Design and Participants

This open-label Phase 1 clinical trial was approved by the institutional review board of Baylor College of Medicine, and by the US Food and Drug Administration (ClinicalTrials.gov identifier: NCT01109095). Written informed consent was obtained from patients or guardians before enrollment on the study. This trial utilized the modified continual assessment method (mCRM) with a cohort size of 3 patients per dose level in order to determine the maximum tolerated dose (MTD). Patients received one or more intravenous infusions of autologous HER2/CMV T-cells at five dose levels (1×10⁶/m², 3×10⁶/m², 1×10⁷/m², 3×10⁷/m², and 1×10⁸/m²). All patients were infused between Jul. 21, 2011 and Apr. 21, 2014. Follow up continued until Jul. 1, 2015.

Patients with histologically confirmed GBM (WHO grade IV glioma) that was either recurrent or progressive after first-line therapy were enrolled on the study after the diagnosis was confirmed by two independent pathologists. All patients had magnetic resonance imaging (MRI) to assess the disease status before T-cell infusion. Eligibility criteria included HER2-positive GBM, CMV-seropositivity, normal left ventricular ejection fraction (LVEF), Karnofsky/Lansky performance score >50 and life expectancy >6 weeks at the time of T-cell infusion. Patients had to have completed (and recovered from) cytotoxic therapy at least 4 weeks before T-cell infusion. One exception was temozolomide (TMZ); because of its extremely short half-life, patients were allowed to receive TMZ up to two days prior to T-cell infusion. Patients with HIV seropositivity, inadequate liver function and renal insufficiency were excluded from the study.

Procedures

HER2 positivity of patient's GBMs and the presence of CMV antigens (pp65, IE1) was determined by immunohistochemistry.^{24,25} HER2/CMV T-cells were manufactured according to current Good Manufacturing Practice (GMP) guidelines using autologous patient's peripheral blood mononuclear cells (PBMCs) that were obtained with a standard blood draw. CMV-specific T-cells were generated as part of a tri-virus approach, which produces a single cell line containing T-cells specific for CMV, Epstein Barr Virus (EBV) and Adenovirus (Ad), as previously described.^{24,26} T cells were transduced with the HER2-specific CAR using clinical grade SFG-FRP5-CD28.ζ retroviral vector as previously described.²⁷ HER2/CMV T-cells were tested for sterility, HLA-identity, and immunophenotype. HER2- and CMV-specificity were determined using cytotoxicity and Elispot assays, respectively, as previously described and as detailed below.

Toxicity was monitored using the NCI Common Terminology Criteria for Adverse Events (CTCAE, version 4.X). Peripheral blood samples were obtained from all prior to each T-cell infusion and then at regular predetermined time points to evaluate for infusion related toxicity, and perform correlative studies as detailed in the Supplemental Method section. Clinical response to T-cell infusion was evaluated by performing MRIs prior to, and 6 weeks post T-cell infusion. Disease response was defined as complete response (CR; absence of initial marker of disease), partial response (PR; reduction in disease marker by at least 50%), stable disease (SD; no change in disease marker) or no response (increase in the measurements of disease marker). Patients with evidence of clinical benefit in the form of SD or response at their 6 week evaluation were eligible to receive additional doses of T-cells.

Generation of HER2/CMV T-Cells

PBMCs were transduced with a clinical grade adenoviral vector encoding the immunodominant CMV pp65 antigen (Ad5f35pp65) after an overnight adherence step. Starting on day 10 post transduction, the cells were re-stimulated weekly with irradiated autologous EBV-transformed lymphoblastoid cell lines transduced with the same Ad5f35pp65 vector (Ad5f35pp65-LCL). Three to 4 days after the 2nd Ad5f35pp65-LCL stimulation T-cells were transduced with a clinical grade retroviral vector encoding a HER2-specific CAR, consisting of a murine scFv FRP5, a short hinge, a CD28 transmembrane domain, and a CD28.ζ signaling domain.27 HER2/CMV T-cells were cryopreserved 7 to 10 days after the 4th stimulation.

Outcomes

The primary objective of this study was to assess the feasibility of generating autologous HER2/CMV T-cells from GBM patients, to define the MTD, and to determine treatment-related toxicities. Secondary objectives were to measure the expansion and persistence of infused T-cells in the peripheral blood, their ability to enhance CMV-specific immunity, and their anti-GBM activity.

Statistical Methods

Safety data were described by the number and proportion of patients who had treatment-related toxicity. Progression free survival (PFS) and OS were analyzed using Kaplan-Meier methods. Transgene expression and Elispot assays were summarized over time using descriptive statistics. The significance between groups was determined by t-test or using the Fisher's exact test. A p-value less than 0.05 was considered statistically significant. Univariate or multivariate logistics and Cox regression models were used to analyze the associations of potential risk factors with response and survival outcomes, respectively.

Supplementary Methods

Generation of HER2/CMV T-Cells

Autologous HER2-CAR modified CMVpp65-specific T-cells (HER2/CMV T-cells) were manufactured from peripheral blood mononuclear cells (PBMCs) according to current Good Manufacturing Practice (cGMP) guidelines as previously described.¹ Peripheral blood mononuclear cells (PBMCs) were transduced with a clinical grade adenoviral vector encoding the immunodominant CMV pp65 antigen (Ad5f35pp65) after an overnight adherence step. 1 Starting on day 10 post transduction, the cells were re-stimulated weekly with irradiated autologous EBVtransformed lymphoblastoid cell lines (LCLs) transduced with the same Ad5f35pp65 vector (Ad5f35pp65-LCL). Three to 4 days after the 2nd Ad5f35pp65-LCL stimulation T-cells were transduced with a clinical grade retroviral vector encoding a HER2-specific CAR, consisting of a murine scFv FRP5, a short hinge, a CD28 transmembrane domain, and a CD28.ζ signaling domain.^{2,3} HER2/CMV T-cells were cryopreserved 7 to 10 days after the 4th stimulation.

Flow Cytometry

A FACSCalibur instrument (Becton Dickinson, San Jose, Calif.) and CellQuest software (Becton Dickinson) was used for flow cytometric analysis.3 Monoclonal antibodies (MAbs) were obtained from Becton Dickinson and included anti-CD3, -CD4, -CD8, -CD16, -CD19, -CD56, -CD62L, -CCR7, -TCRα/β, and -TCRγ/δ. HER2-CAR expression was detected with a murine scFV-specific MAb (Jackson ImmunoResearch Laboratories). Negative controls included isotype antibodies.

Real-Time PCR Assay

A FRP5-specific primer and TaqMan probe (Applied Biosystems) were used to detect HER2-CAR T cells.³ DNA was extracted with the QIAamp DNA Blood Mini Kit (Qiagen) and qPCR was performed in triplicates using the ABI RPISM 7900HT Sequence Detection System (Applied Biosystems). The baseline range was set at cycles 6-15, with the threshold at 10 SDs above the baseline fluorescence. To generate DNA standards, serial dilution of DNA plasmids were established encoding each specific cassette.

Enzyme-Linked Immunospot (Elispot) Assay

The frequency of antigen-specific T cells in the HER2/CMV T-cell product and peripheral blood of patients was measured using interferon-γ (IFN-γ) Elispot assays as previously described. ^{1,4} Briefly, HER2/CMV T-cells or PBMCs were stimulated with overlapping peptide mixes for pp65, IE1, hexon, and penton. Peptidemixes contained 15 amino-acid peptides covering the entire length of the corresponding protein with an 11 amino-acid overlap (pepmixes; JPT Peptide Technologies, Berlin, Germany). Media (no peptide) served as negative control, and Phytohemagglutinin (PHA, Sigma) as positive control. Developed Elispots were analyzed by ZellNet Consulting (New York, N.Y.). Spot-forming cells (SFCs) were calculated and expressed as SFC per 10⁵ cells for T-cell products and 2×10⁵ cells for PBMCs.

Cytotoxicity Assay

Cytotoxic activity of HER2/CMV T-cells against targets was determined by standard 51Cr release assay.2 1×10⁶ target cells were labeled with 50 μCi ⁵¹Cr and incubated for 1 hour. Targets were then washed and 5×10³ cells were co-cultured with effector T cells at different effector to target (E:T) ratios. Supernatants were analyzed with a Packard Cobra Quantum gamma counter Model E 5010 (Perkin Elmer, Shelton Conn.) reader after 4 hour incubation. Lysis was calculated as previously described.

Immunohistochemistry (IHC)

Formalin-fixed, paraffin embedded sections (6 m) of GBM were processed as previously described,4-6 and stained with a phospho-HER2 MAb (CB 11, Abcam, Cambridge, Mass.) for HER2 detection, or a CMV IE1 MAb (1:100; Chemicon, Temecula, Calif., USA) and CMV pp65 Mab (1:40; Leica Microsystems Inc., Bannockburn, Ill., USA) for detection of the respective CMV proteins. All slides were counterstained in Harris hematoxylin. Known HER2-expressing breast cancer samples and CMV-infected lung samples were used as a positive controls, respectively. Slides only stained with secondary MAb served as negative controls.

References for Supplementary Methods
1. Leen A M, Myers G D, Sili U, et al. Monoculture-derived T lymphocytes specific for multiple viruses expand and produce clinically relevant effects in immunocompromised individuals. Nature medicine 2006; 12(10): 1160-6.
2. Ahmed N, Salsman V S, Kew Y, et al. HER2-specific T cells target primary glioblastoma stem cells and induce regression of autologous experimental tumors. ClinCancer Res 2010; 16(2): 474-85.
3. Ahmed N, Brawley V S, Hegde M, et al. Human Epidermal Growth Factor Receptor 2 (HER2) -Specific Chimeric Antigen Receptor-Modified T Cells for the Immunotherapy of HER2-Positive Sarcoma. Journal of clinical oncology: official journal of the American Society of Clinical Oncology 2015; 33(15): 1688-96.
4. Ghazi A, Ashoori A, Hanley P J, et al. Generation of polyclonal CMV-specific T cells for the adoptive immunotherapy of glioblastoma. JImmunother 2012; 35(2): 159-68.
5. Wakefield A, Pignata A, Ghazi A, et al. Is CMV a target in pediatric glioblastoma? Expression of CMV proteins, pp65 and IE1-72 and CMV nucleic acids in a cohort of pediatric glioblastoma patients. Journal of neuro-oncology 2015.
6. Ahmed N, Ratnayake M, Savoldo B, et al. Regression of experimental medulloblastoma following transfer of HER2-specific T cells. Cancer Res 2007; 67(12): 5957-64.

Results

Between Jul. 25, 2011 to Apr. 21, 2014, 17 patients (8 female, 9 male) were enrolled on the study (Table 4). Ten of 17 patients were ≧18 years of age (median 57 years; range: 30-69 years). Seven patients were <18 years of age (median 14 years; range: 10-17 years).

4: Patient Characteristics

| | | Prior Treatment | | | | |
|---|---|---|---|---|---|---|
| | Age | | | | | Time to |
| | (years)/ | | | | Investigational | T-cell Therapy |
| UPN | sex | Surgery | XRT + TMZ | Salvage Therapies | Agents | from dx (mths) |
| 01 | 42.8/F | Yes, x3 | Yes | (1) TMZ + Hydrox (2) TMZ | (1) TMZ + Iniparib | 27.2 |
| | | | | (3) CCNU + Bev (4) TMZ + | (2) Carbo + Iniparib | |
| | | | | Hydrox + Bev (5) Irino + Bev | | |
| 02 | 59.3/M | Yes, at dx | Yes | (1) Bev | None | 12.4 |
| 03 | 29.6/M | Yes, at dx | Yes | (1) BCNU (2) Bev | (1) Veliparib | 16.0 |
| 04 | 17.1/M | Biopsy only | Yes | None | None | 7.3 |
| 05 | 62.2/M | Yes, x2 | Yes | (1) Irino + Bev | None | 27.2 |
| 06 | 59.4/F | Yes, x2 | Yes | (1) TMZ + Accutane + | (1) Toca 511 | 20.3 |
| | | | | Verapamil + Metformin + | (2) Bev + EGFRvIII | |
| | | | | Tamox | vaccine | |
| 07 | 60.9/F | Yes, at dx | Yes | (1) Bev + Carbo | (1) Imatinib mesylate | 13.3 |
| 08 | 10.6/M | Yes, at dx | XRT, No TMZ | None | None | 7.3 |
| 09 | 63.5/M | Yes, x2 (STR | Yes | None | (1) Veliparib | 12.8 |
| | | then GTR) | | | | |
| 10 | 50/M | Yes, at dx | Yes | (1) Paclitaxel | None | 13.2 |
| 11 | 62.7/M | Yes, at dx | Yes | None | None | 11.3 |
| 12 | 13.1/M | Yes, x2 | Yes | (1) Bev | None | 6.2 |
| 13 | 69.3/F | Yes, at dx | Yes | None | None | 17.0 |
| 14 | 14.4/M | Yes, STR x2 | XRT, No TMZ | None | None | 16.7 |
| 15 | 14.4/F | Yes, x4 | XRT, No TMZ | (1) Vori (2) Dasatinib | (1) 5-FU + IFNα2b | 5.9 |
| 16 | 10/F | Yes, x2 | Yes | None | None | 9.2 |
| 17 | 16.2/F | Yes, at dx | Yes | (1) TMZ + CCNU | None | 12.9 |

UPN: Unique patient number
Hydrox: Hydroxychloroquine
BCNU: Carmustine
Toca 511: Vocimagene amiretrorepvec
5-FU: Fluorouracil
dx: Diagnosis
CCNU: Lomustine
Tamox: Tamoxifen
IFN: Interferon
mths: Months
XRT: Radiation therapy
Bev: Bevacizumab
Carbo: Carboplatin
STR: Subtotal resection
TMZ: Temozolomide
Irino: Irinotecan
Vori: Vorinostat
GTR: Gross total resection

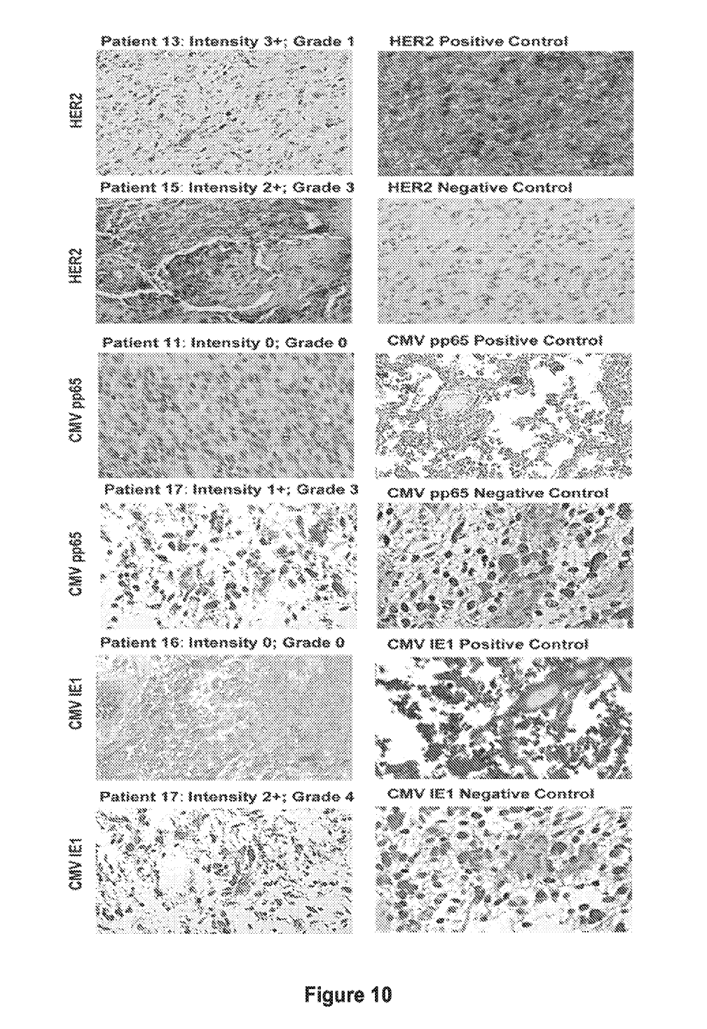

All patients (except patient 1) were CMV seropositive. All patients had recurrent or progressive GBM at the time of T-cell infusion and HER2 positivity was confirmed by IHC (FIG. 10, Table 5).

TABLE 5
Expression of HER2, CMV pp65, and
CMV IE1 in GBMs of study patients

| | | | CMV | | | |
|---|---|---|---|---|---|---|
| | HER2 | | pp65 | | IE1 | |
| UPN | Intensity | Grade | Intensity | Grade | Intensity | Grade |
| 1 | 2+ | 2 | — | — | — | — |
| 2 | 2+ | 1 | 0 | 0 | 1+ | Grade 1 |
| 3 | 1+ | 1 | NE | NE | NE | NE |
| 4 | 2+ | 4 | 0 | 0 | 0 | 0 |
| 5 | 3+ | 2 | 0 | 0 | 0 | 0 |
| 6 | 2+ | 2 | NE | NE | 0 | 0 |
| 7 | 2+ | 2 | 1+ | 1 | 1+ | 1 |
| 8 | 1+ to 2+ | 1 | 1+ | 1 | 1+ | 1 |
| 9 | 3+ | 2 | 0 | 0 | 1+ | 1 |
| 10 | 2+ | 2 | 0 | 0 | 1+ | 1 |
| 11 | 3+ | 2 | 0 | 0 | 0 | 0 |
| 12 | 2+ | 1 | 0 | 0 | 0 | 0 |
| 13 | 3+ | 1 | 2 | 2 | 0 | 0 |
| 14 | 2+ | 2-3 | 0 | 0 | 0 | 0 |
| 15 | 2+ | 1 | 1+ | 4 | 1+ | 4 |
| 16 | 2+ | 3 | 0 | 0 | 0 | 0 |
| 17 | 3+ | 2 | 1+ | 3 | 2+ | 4 |

Intensity: 0 to 3+ based on positivity of control slides
Grade (percentage of positive tumor cells): 0 = none, 1 = 1-25%, 2 = 26-50%, 3 = 51-75%, 4 = 76-100%
NE: not evaluable

All patients (except patient 4) had surgical resections followed by radiation therapy (RT) with concomitant TMZ. Eight of 17 patients (47%) had undergone 2 or more surgical resections. All adult patients and three of seven children had received TMZ for ≧6 months. Patients 2 and 3 had received salvage RT/surgery. Ten patients (59%) had failed 1-5 lines of additional salvage therapies and six of 17 patients had received investigational therapies prior to study enrollment. Median time to T-cell infusion from diagnosis was 13•3 months (range: 3•5 to 27•7 months).

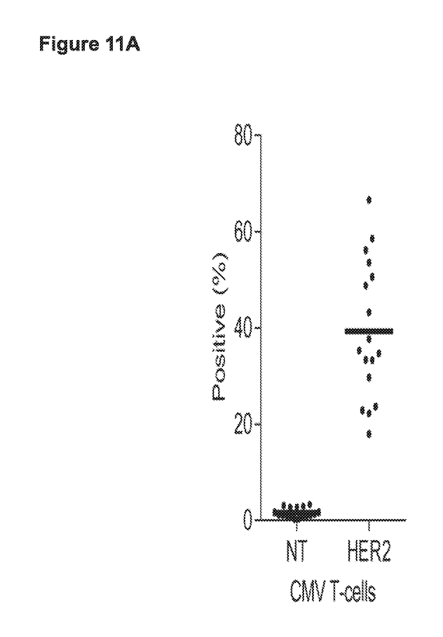

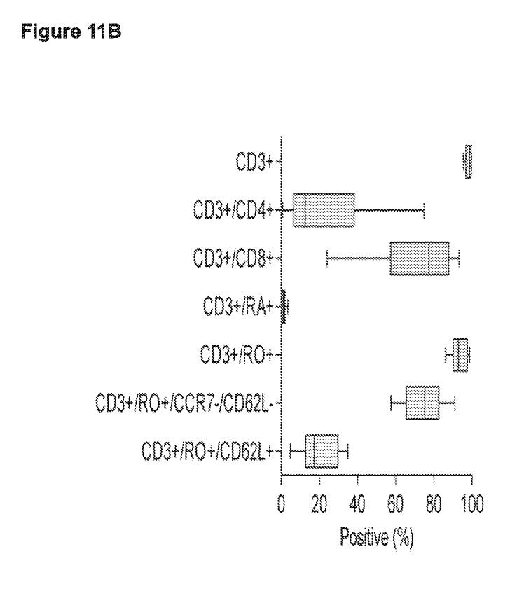

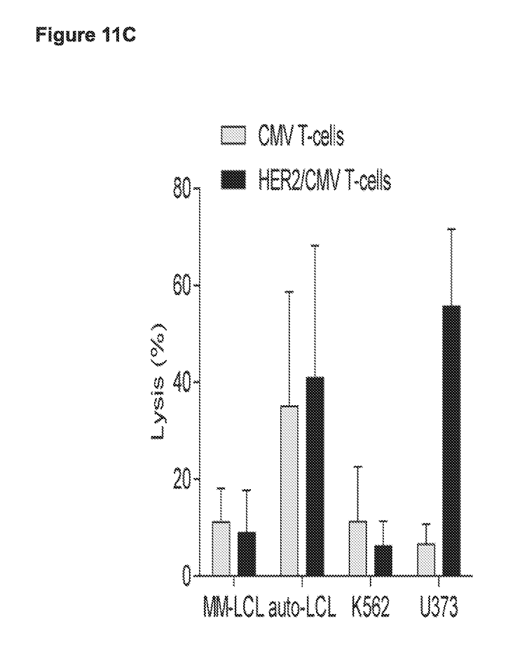

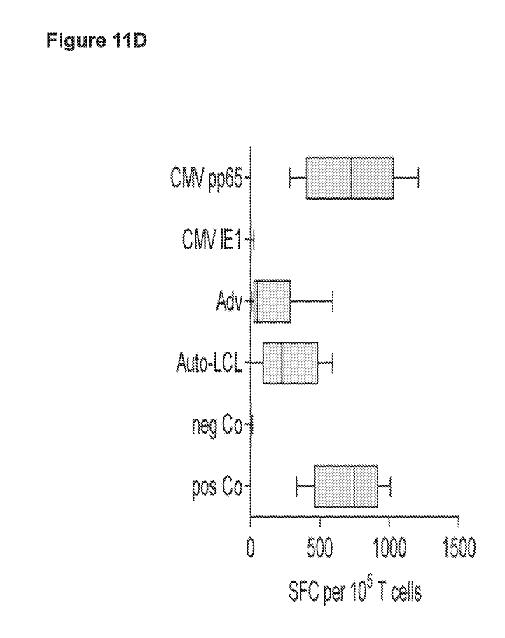

Autologous HER2/CMV T-cell products were successfully generated for all patients. The mean HER2-CAR transduction efficiency was 39% (range 18-67%; FIG. 11A). Cell products contained CD3+/CD8+(mean 71%; range 16-97%) and CD3+/CD4+(mean: 24%; range: 0.3-88%) T-cells. The majority of T cells had a memory phenotype (CD45RO+; mean: 94%; range: 86-100%) consisting of effector (CD45RO+/CCR7−/CD62L−; mean: 74%; range: 42-94%) as well as central (CD45RO+/CD62L+; mean: 20%; range: 2-49%) memory T-cell subsets (FIG. 11B). In standard cytotoxicity assays, HER2/CMV T-cells had significant cytotoxicity against the HER2-positive glioma cell line U373 in contrast to unmodified CMV Tcells. Only background killing was observed against HER2-negative K562. While HER2/CMV T-cell products of all 16 CMV-seropositive GBM patients contained CMV-, adenovirus (Adv)-, and EBV-specific T cells, the dominant virus-specific reactivity was directed against pp65 as judged by IFN-γ Elispot assays (FIG. 11C, 11D).

Seventeen patients received a total of 30 infusions, with 6 patients receiving multiple infusions (3 patients received 2, 1 patient 3, 1 patient 4, 1 patient 6; Table 6).

TABLE 6
Patient Outcomes

| | | | | Time to | Survival | | |
|---|---|---|---|---|---|---|---|
| | | | Disease | Progression | (in months) | | |
| | Disease at T-cell infusion; | T-cell | Reponses | (months from | From first | From | |
| UPN | measurement | dose/m² | (6 weeks) | first infusion) | T-cell infusion | diagnosis | Outcome |
| 01 | Genu of the corpus callosum and | 1 × 10⁶ | SD | 4.4 | 27.8 | 55.0 | DOD |
| | left forceps minor; irregular shape | 3 × 10⁶ | | | | | |
| | | 1 × 10⁷ | | | | | |
| | | 3 × 10⁷ | | | | | |
| 02 | Right parietal lobe; irregular | 1 × 10⁶ | PD | 4.0 | 4.0 | 16.4 | DOD |
| | shape | | | | | | |
| 03 | Left temporal lobe; 3 cm | 1 × 10⁶ | PD | 2.1 | 15.5 | 31.5 | DOD |
| 04 | Right thalamic lesion: 4 × 3 cm | 1 × 10⁶ (x2) | PR | 9.2 | 26.9 | 34.2 | DOD |
| 05 | Left frontal lobe; 4.7 × 3.8 cm | 3 × 10⁶ | PD | 3.6 | 3.7 | 30.9 | DOD |
| 06 | Left parietal lobe; 4.6 × 3.8 cm | 3 × 10⁶ | SD | 2.3 | 2.4 | 22.7 | Death from |
| | | | | | | | peritoneal bleed |
| 07 | Corpus callosum; 2 × 0.7 cm | 3 × 10⁶ | PD | 1.4 | 6.9 | 20.3 | DOD |
| 08 | Right frontoparietal cortex; | 1 × 10⁷ | SD | no | 28.6 | 35.9 | Alive |
| | stellate | | | progression | | | |
| 09 | Temporopaietal; 1 cm rim | 1 × 10⁷ (x6) | SD | no | 28.4 | 41.2 | Alive |
| | enhancement | | | progression | | | |
| 10 | Right parietal, right pulvinar | 1 × 10⁷ | PD | 0.8 | 10.9 | 24.1 | DOD |
| | region, right periventricular, | | | | | | |
| | anterior insular cortex | | | | | | |
| | (multifocal) | | | | | | |
| 11 | Rim enhancement; 1 cm thick | 3 × 10⁷ | SD | unknown | 7.9 | 19.2 | DOD |
| 12 | Rim enhancement; 1 cm thick | 3 × 10⁷ | PD | 1.1 | 2.7 | 8.9 | DOD |
| 13 | Frontal lobe rim enhancement; 1 | 3 × 10⁷ (x3) | SD | no | 23.7 | 40.7 | Alive |
| | cm thick | | | progression | | | |
| 14 | Left temporal lobe; 3.2 × 1.5 cm | 3 × 10⁶ | PD | 1.2 | 6.1 | 22.8 | DOD |
| 15 | Bilateral frontal lobe butterfly | 1 × 10⁸ (x2) | SD | 2.7 | 6.4 | 12.3 | DOD |
| | lesion; 8.3 × 6.7 × 6.5 cm | | | | | | |
| 16 | Right temporal lobe lesion; | 1 × 10⁸ (x2) | PD | 1.3 | 7.8 | 17.0 | DOD |
| | 2.9 × 1.6 cm | | | | | | |
| 17 | Left thalamus; 2.2 × 1.2 cm | 1 × 10⁸ (x2) | PD | 3.5 | 11.3 | 24.2 | DOD |
| | lesion | | | | | | |

PR: Partial response
PD: Progressive disease
SD: Stable disease
DOD: Died of disease

None of the patients had adverse events related to the T-cell infusion; study-unrelated Grade 2-4 adverse events are summarized in Table 7. At 6 weeks post infusion, cardiac function studies showed unchanged LVEFs from pre-infusion values.

TABLE 7
Unrelated adverse events within the first
6 weeks post HER2/CMV T-cell infusion

| | Grade 2 | | Grade 3 | | Grade 4 | |
|---|---|---|---|---|---|---|
| | No. of | | No. of | | No. of | |
| Adverse Event | Patients | % | Patients | % | Patients | % |
| Hematologic Toxicities | | | | | | |
| Anemia | 1 | 5.9 | | | | |
| Lymphopenia | 7 | 41.2 | 2 | 11.8 | | |
| Neutropenia | 2 | 11.8 | | | 1 | 5.9 |
| Thrombocytopenia | 1 | 5.9 | | | | |
| Non-hematologic | | | | | | |
| Toxicities | | | | | | |
| General | | | | | | |
| Anorexia | 1 | 5.9 | | | | |
| Fatigue | | | 1 | 5.9 | | |
| Somnolence | 1 | 5.9 | | | | |
| Weakness | 2 | 11.8 | 1 | 5.9 | | |
| HEENT | | | | | | |
| Eye paralysis, Lateral | 1 | 5.9 | | | | |
| GI | | | | | | |
| Nausea | 2 | 11.8 | | | | |
| Diarrhea | 1 | 5.9 | | | | |
| Constipation | 1 | 5.9 | | | | |
| Vomiting | 2 | 11.8 | | | | |
| Cardiac | | | | | | |
| Bradycardia | 1 | 5.9 | | | | |
| Respiratory | | | | | | |
| Atelectasis | 1 | 5.9 | | | | |
| Pain | | | | | | |
| Extremity | 1 | 5.9 | | | | |
| Bone | 1 | 5.9 | | | | |
| Myalgias | 1 | 5.9 | | | | |
| Musculoskeletal | | | | | | |
| Edema, localized | 1 | 5.9 | | | | |
| Fracture | 1 | 5.9 | | | | |
| CNS | | | | | | |
| Headache | 1 | 5.9 | 2 | 11.8 | | |
| Seizure | 2 | 11.8 | | | | |
| Gait Disturbance | 2 | 11.8 | | | | |
| Memory Impairment | 1 | 5.9 | | | | |
| Tremors | 1 | 5.9 | | | | |
| Cerebral Edema | | | | | 1 | 5.9 |
| Hydrocephalus | | | 1 | 5.9 | | |
| Infectious | | | | | | |
| UTI | 1 | 5.9 | | | | |
| Laboratory | | | | | | |
| ALT | 1 | 5.9 | | | | |
| AST | 1 | 5.9 | | | | |
| Hyperbilirubinemia | 1 | 5.9 | | | | |
| Hyperkalemia | 1 | 5.9 | | | | |
| Hypernatremia | 1 | 5.9 | | | | |
| Hyponatremia | 1 | 5.9 | 1 | 5.9 | | |

ALT: elevated alanine aminotransferase
AST: elevated aspartate aminotransferase
HEENT: head, ears, eyes, nose, and throat

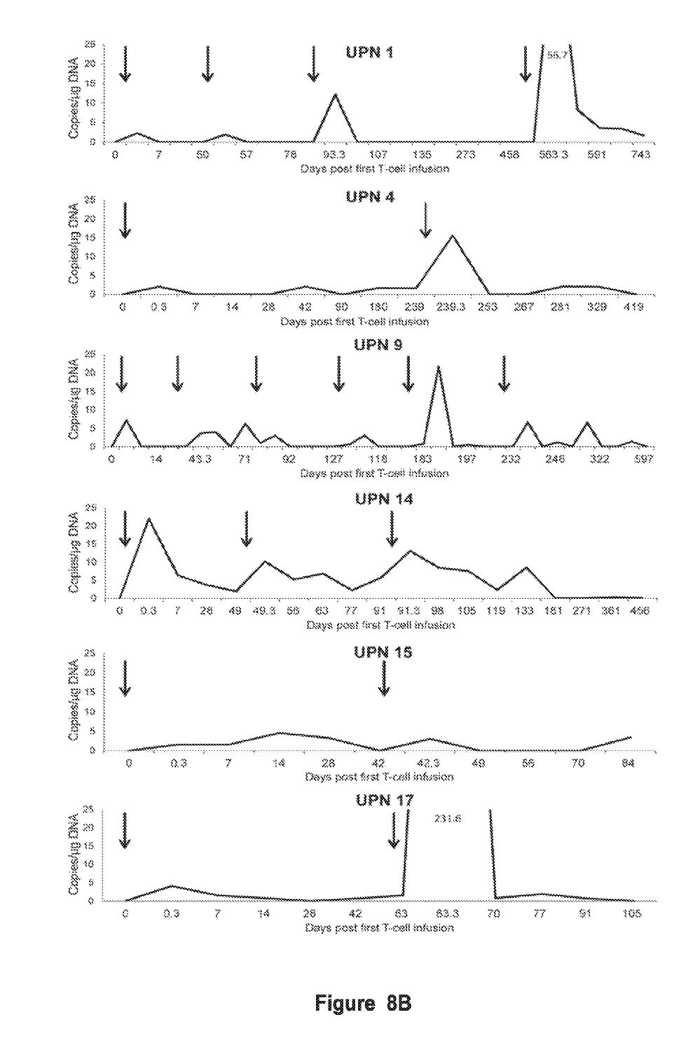

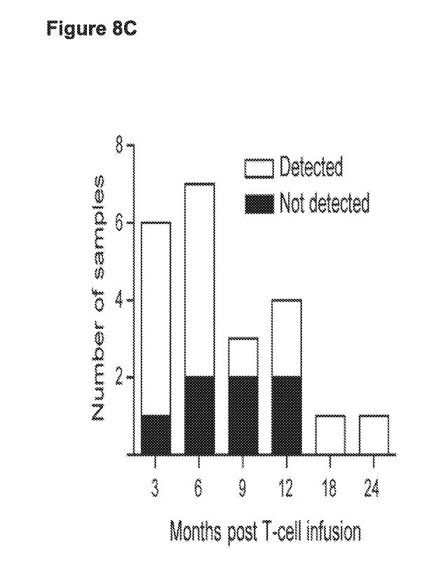

HER2/CMV T-cells were detected by using quantitative real-time polymerase chain reaction (qPCR) in all patients post infusion. Fifteen out of 17 patients had their highest frequency of HER2/CMV T-cells 3 hours post infusion (mean: 7•8 copies/μg DNA, range: 1•4 to 27•8 copies/μg DNA), 1 patient at 1 week (2•0 copies/μg DNA), and 1 patient at 2 weeks (7•2 copies/μg DNA; FIGS. 8A and 8B). At 6 weeks post infusion HER2/CMV T cells were present in seven of 15 patients (mean: 2•0 copies/μg DNA, range: 0•7 to 3•8 copies/μg DNA). HER2/CMV T cells were detected in one of six samples analyzed at three months, in two of seven samples analyzed at six months, in two of three samples analyzed at nine months, and in two of four samples analyzed at 12 months, and were not detectable in one sample analyzed at 18 and 24 months (FIG. 8C). Thus, while there was no evidence of HER2/CMV T-cell expansion in 15 of 17 patients using qPCR, these cells could be maintained in the peripheral circulation for up to 12 months with repeat infusions.

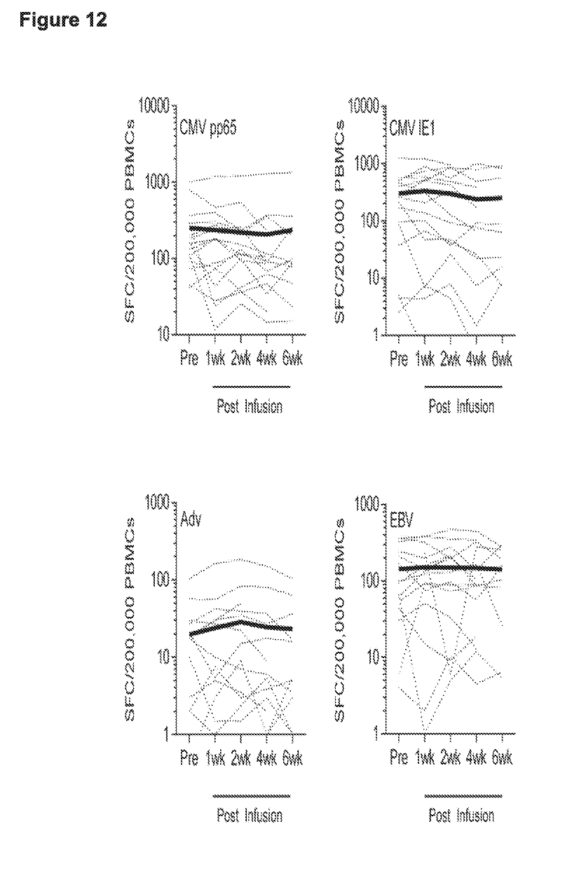

To determine the frequency of CMV pp65-specific T cells in the peripheral blood we performed IFN-γ Elispot assays using CMV pp65 peptide mixes (pepmixes) as stimulator. Additionally, Adenovirus hexon/penton pepmixes and autologous lymphoblastoid cell line (LCL; EBV immortalized B cells) were used as stimulators to detect Adeno- and EBV-specific T cells, respectively. As a control, the frequency of T-cells specific for the CMV antigen IE1 was measured. There was no significant decline or increase in the frequency of pp65-, hexon/penton-, and LCL-specific T cells after HER2/CMV T-cell infusion; in addition, there was no change in endogenous, IE1-specific T-cell immunity (FIG. 12).

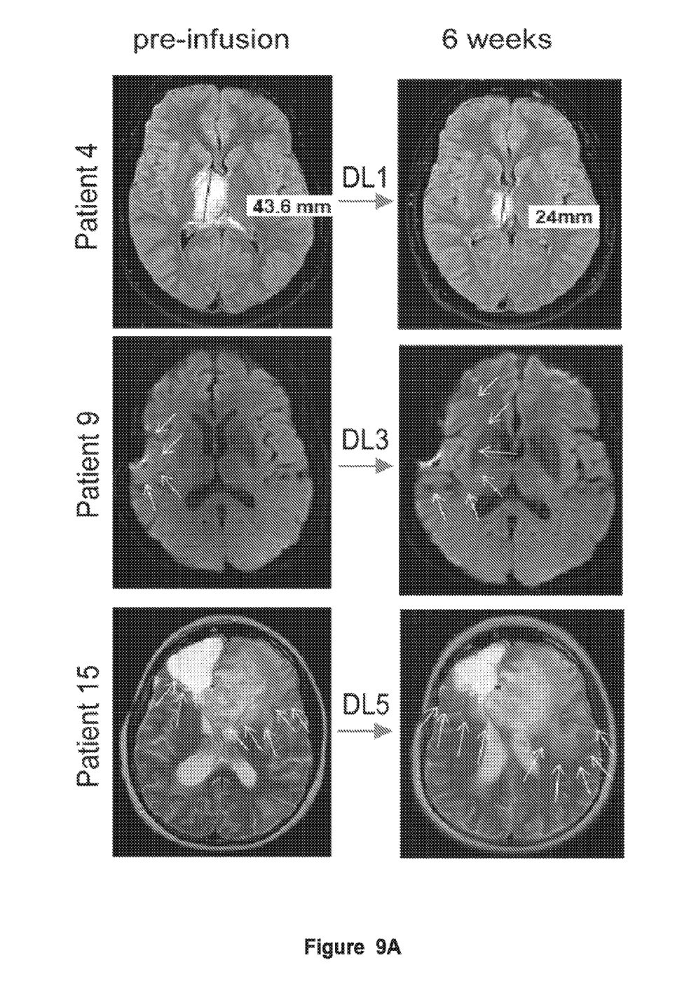

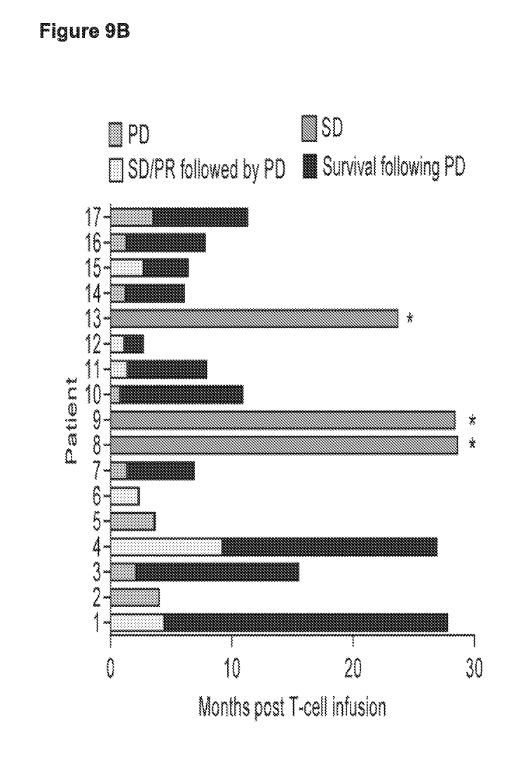

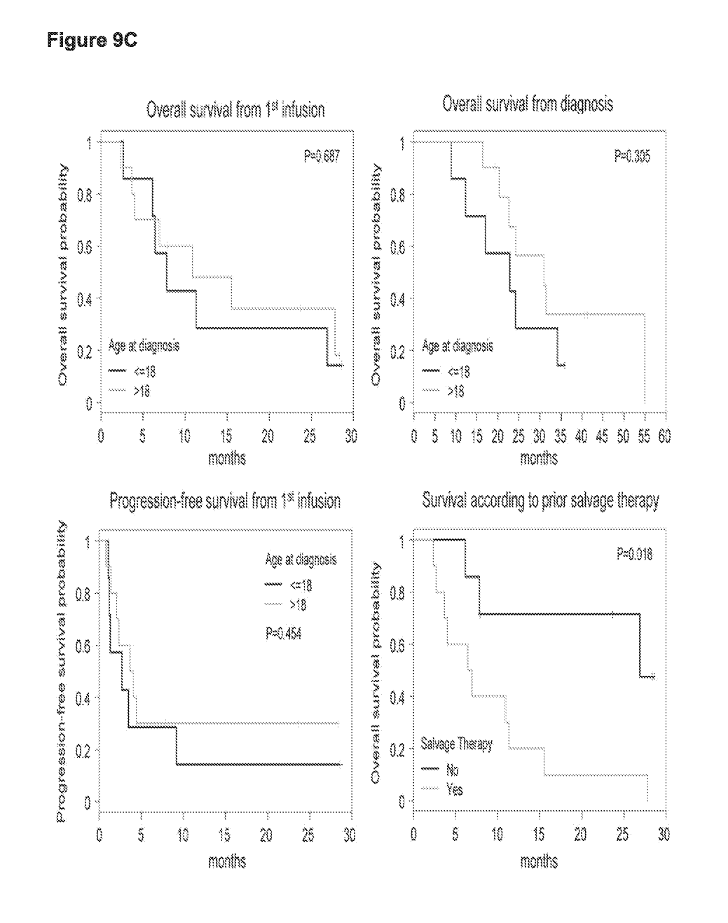

To evaluate the anti-GBM activity of HER2/CMV T-cells, brain MRIs were done 6 weeks post T-cell infusions (FIG. 9A). Patient 14 received chemotherapy within the first 6 weeks of T-cell infusion and was excluded from the response analysis. Of 16 evaluable patients, one patient (6%; patient 4) had a PR and 7 other patients (41%) had SD for 2•3 to >29 months after the first T-cell infusion (Table 6). Patient 4, a 17-year old male with an unresectable right thalamic GBM (4•6 cm), received 1×10⁶/m² HER2/CMV T-cells and had PR that lasted for 9•2 months (FIG. 9A). He then had SD after a second infusion on the same dose level, and survived for 27•3 months from the first infusion (Table 7). Three patients (18%; patients 8, 9 and 13) are alive with SD for 29, 28•8 and 24 months of follow up. Eight patients had PD based on RECIST criteria. Despite disease progression 5 patients survived for ≧5•5 months (range: 5•5 to 13•6 months; FIG. 9B).

For the entire study cohort, the median time to progression was 3•5 months; median OS was 11•6 months post first T-cell infusion and 24•8 months post diagnosis (FIG. 9C). There was no significant difference in PFS and OS between pediatric (<18 years at diagnosis) and adult patients (p=0•4; FIG. 9C). Cox regression analysis showed that patients who did not receive salvage therapy prior to infusion had a significantly longer OS probability (27 months) compared to those infused after prior salvage therapy (7 months; p=0.018; FIG. 9C). Univariate and multivariate analysis of other metrics did not correlate with response or survival outcomes (Table 8).

TABLE 8
Univariate Cox regression analysis

| | PFS | | OS | |
|---|---|---|---|---|
| | Hazard ratio | | Hazard ratio | |
| Variable | (95% CI) | P value | (95% CI) | P value |
| Age at diagnosis | | | | |
| ≤18 years | 1.519 | 0.457 | 1.252 | 0.688 |
| | (0.505-4.567) | | (0.419-3.743) | |
| >18 years | 1 | | 1 | |
| Sex | | | | |
| Female | 1.384 | 0.565 | 1.312 | 0.629 |
| | (0.457-4.189) | | (0.437-3.941) | |
| Male | 1 | | 1 | |
| Salvage therapy | | | | |
| No | 1 | | 1 | |
| Yes | 6.24 | 0.023 | 4.302 | 0.029 |
| | (1.291-30.161) | | (1.16-15.958) | |
| Time to T-cell | | | | |
| therapy from dx | | | | |
| ≤14 months | 1 | | 1 | |
| >14 months | 1.171 | 0.783 | 1.27 | 0.678 |
| | (0.381-3.6) | | (0.41-3.932) | |
| HER2 expression | | | | |
| grade* | | | | |
| <2 | 1 | | 1 | |
| ≥2 | 1.389 | 0.586 | 1.203 | 0.761 |
| | (0.426-4.532) | | (0.367-3.944) | |
| HER2 expression | | | | |
| intensity** | | | | |
| ≥3 | 1 | | 1 | |
| <3 | 4.203 | 0.064 | 2.788 | 0.183 |
| | (0.922-19.16) | | (0.615-12.635) | |
| Number of | | | | |
| T-cell infusions | | | | |
| Single | 1.889 | 0.273 | 2.265 | 0.161 |
| | (0.605-5.894) | | (0.723-7.092) | |
| Multiple | 1 | | 1 | |

*Grade (cells positive): 1 = 1-25%, 2 = 26-50%, 3 = 51-75%, 4 = 76-100%
**Intensity: 1+ to 3+ based on positivity of control slides

### Significance of Certain Embodiments

In this phase 1 dose-escalation study, the safety of autologous HER2/CMV Tcells was established in 17 patients with recurrent/progressive GBM. While HER2/CMV T-cells did not expand, they were detectable in the peripheral blood for up to 12 months. Eight patients had clinical benefit as defined by PR (n=1) and SD (n=7). The median OS was 11•6 months post T-cell infusion and 24.8 months from diagnosis. Three patients with SD were alive at the time of last follow-up with no disease progression.

CAR T-cell therapies are an attractive strategy to improve the outcomes for patients with GBM. So far, only one study has been published in which 3 GBM patients received an intratumoral injection of T cells that were genetically modified with a first generation IL13Rα2-specific CAR.¹³ Local injections were well tolerated and two of three patients had a transient clinical response.¹³ In this study HER2/CMV T-cells were infused intravenously, since T-cells can traffic to the brain after intravenous injections as evidenced by clinical responses after the adoptive transfer of EBV-specific T-cells for central nervous system post-transplant lymphoproliferative disease (CNS-PTLD)²⁸, responses to tumor infiltrating lymphocytes (TILs) for melanoma brain metastasis²⁹ and isolation of CD19 CAR T cells from the cerebrospinal fluid of patients with CNS B-precursor leukemia.

Infusion of up to 1×10⁸/m² HER2/CMV T-cells was well tolerated without evident toxicities, confirming a previous study, in which CD3/CD28-activated T-cells has been infused that expressed the same CAR.¹⁹ While HER2/CMV T-cells were detectable for up to 12 months post infusion there was no observed significant in vivo expansion in the peripheral blood of infused patients as judged by qPCR. In addition, there were no significant changes in the frequency of CMV-specific T-cell responses post infusion. The findings are in agreement with previous studies in which GBM patients received unmodified CMV-specific T-cells, 30 or neuroblastoma patients received EBV-specific T-cells genetically modified to express GD2-CARs (GD2-CAR/EBV T-cells).¹² Lack of in vivo expansion of CMV- and EBV-specific T cells in both studies contrast to the significant expansion of these cells in hematopoietic stem cell transplant recipients, who are severely lymphodepleted and have reactivation of the corresponding virus.^{31,32} GBM patients on this study had a normal absolute lymphocyte counts (ALCs, mean: 1130; range: 421-2318) at the time of T-cell infusion. Thus lymphodepleting chemotherapy and/or the provision of viral antigens in the form of vaccines is useful, in at least one embodiment, to increase the in vivo expansion of adoptively transferred HER2/CMV T-cells in GBM patients. Indeed, lymphodepleting chemotherapy has shown to be critical for the robust expansion of CD19-CAR T-cells in patients with hematological malignancies,⁸ and vaccines have been used successfully to boost the expansion of CAR/virus-specific T-cells in preclinical models.³³

While there was no observed expansion of HER2/CMV T-cells in the peripheral blood, T-cells could have expanded at GBM sites. At 6 week post T-cell infusion MRIs of 5 patients showed an increase in peri-tumoral edema. While these patients were classified as having PD, it is likely that the imaging changes in some of these patients were due to inflammatory responses, indicative of local T-cell expansion, especially since 5 of these patients survived for >5•5 months. Local inflammatory response, so-called pseudo-progression, has been observed on several immunotherapy studies, especially for GBMs, highlighting the need to develop novel response criteria.⁴ In this regard, the Response Assessment for Neuro-Oncology (RANO) working group recently published their recommendation for immunotherapy studies 34

T-cells were infused that could potentially recognize HER2 and pp65 expressed in GBMs. GBMs of 5 patients were pp65 positive, of whom 2 had PD and 3 SD. Clearly, a larger cohort of patients with pp65-positive GBMs may be utilized to determine if pp65 expression predicts anti-GBM activity of HER2/CMV T-cells.

Outcomes data on post-progression survival (PPS) in GBM patients is limited. One recent Italian study performed a retrospective outcomes analysis of 232 GBM patients, who received second line chemotherapy at disease progression after RT/TMZ. The median PFS was 2•5 months and the median PPS was 8•6 months.³⁵ A randomized controlled phase 2 trial compared the combination of bevacizumab plus lomustine to single agent bevacizumab or lomustine in GBM patients, who had failed frontline therapy.³⁶ While bevacizumab or lomustine were well tolerated, the lomustine dose needed to be reduced in the bevacizumab plus lomustine due to hematological toxicities. Fifty-two patients, who received bevacizumab every 2 weeks and lomustine every 6 weeks, had the best outcome with a median OS of 12 months and an 18-months OS of 20•0%.³⁶ In this cohort of 17 GBM patients, in which 10 patients already had failed 2nd line therapy, we achieved similar outcomes (median OS: 11•6 months; 18-months OS: 29•4%) with a median of 1 (range 1•6) HER2/CMV T-cell infusion without evident toxicities.

In some embodiments, one can improve the anti-GBM activity of HER2/CMV T-cells. Besides lymphodepletion and/or vaccination to enhance the in vivo expansion and persistence of adoptively transferred T-cells, in certain embodiments other manipulation of the immune system is useful, such as blocking inhibitory molecules that are expressed on the cell surface (e.g. PD-L1) or secreted (e.g. TGF-β) by glioma cells.³⁷⁻³⁹ Since antigen expression in GBM is heterogeneous, targeting multiple antigens also has the potential to improve response rates and outcomes.¹⁶

In summary, treatment of recurrent/progressive GBM with HER2/CMV T-cells is feasible and safe, and resulted in clinical benefit in eight of 17 patients. While these data support larger studies, they also highlight the need to improve the anti-GBM activity of HER2/CMV T-cells by augmenting their expansion, function, and persistence.

### REFERENCES

1. Stupp, R., et al. Maintenance Therapy With Tumor-Treating Fields Plus Temozolomide vs Temozolomide Alone for Glioblastoma: A Randomized Clinical Trial. Jama 314, 2535-2543 (2015).
2. Cohen, K. J., et al. Temozolomide in the treatment of high-grade gliomas in children: a report from the Children's Oncology Group. Neuro-oncology 13, 317-323 (2011).
3. Pollack, I. F., et al. Antigen-specific immune responses and clinical outcome after vaccination with glioma-associated antigen peptides and polyinosinic-polycytidylic acid stabilized by lysine and carboxymethylcellulose in children with newly diagnosed malignant brainstem and nonbrainstem gliomas. Journal of clinical oncology: official journal of the American Society of Clinical Oncology 32, 2050-2058 (2014).
4. Okada, H., et al. Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. Journal of clinical oncology: official journal of the American Society of Clinical Oncology 29, 330-336 (2011).
5. Terasaki, M., et al. Phase I trial of a personalized peptide vaccine for patients positive for human leukocyte antigen—A24 with recurrent or progressive glioblastoma multiforme. Journal of clinical oncology: official journal of the American Society of Clinical Oncology 29, 337-344 (2011).
6. Eshhar, Z., Waks, T., Gross, G. & Schindler, D. G. Specific activation and targeting of cytotoxic lymphocytes through chimeric single chains consisting of antibody-binding domains and the gamma or zeta subunits of the immunoglobulin and T-cell receptors. Proceedings of the National Academy of Sciences of the United States of America 90, 720-724 (1993).
7. Grupp, S. A., et al. Chimeric antigen receptor-modified T cells for acute lymphoid leukemia. The New England journal of medicine 368, 1509-1518 (2013).
8. Lee, D. W., et al. T cells expressing CD19 chimeric antigen receptors for acute lymphoblastic leukaemia in children and young adults: a phase 1 dose-escalation trial. Lancet 385, 517-528 (2015).
9. Porter, D. L., Levine, B. L., Kalos, M., Bagg, A. & June, C. H. Chimeric antigen receptor-modified T cells in chronic lymphoid leukemia. The New England journal of medicine 365, 725-733 (2011).
10. M, M. T-cell Therapy Targets for Glioblastoma. (Cancer Discovery May 2015 5:458; Published OnlineFirst Mar. 17, 2015).
11. Pule, M. A., et al. Virus-specific T cells engineered to coexpress tumor-specific receptors: persistence and antitumor activity in individuals with neuroblastoma. Nature medicine 14, 1264-1270 (2008).
12. Louis, C. U., et al. Antitumor activity and long-term fate of chimeric antigen receptor-positive T cells in patients with neuroblastoma. Blood 118, 6050-6056 (2011).
13. Brown, C. E., et al. Bioactivity and Safety of IL13Ralpha2-Redirected Chimeric Antigen Receptor CD8+ T Cells in Patients with Recurrent Glioblastoma. Clinical cancer research: an official journal of the American Association for Cancer Research 21, 4062-4072 (2015).
14. Ahmed, N., et al. HER2-specific T cells target primary glioblastoma stem cells and induce regression of autologous experimental tumors. Clinical cancer research: an official journal of the American Association for Cancer Research 16, 474-485 (2010).
15. Chow, K. K., et al. T cells redirected to EphA2 for the immunotherapy of glioblastoma. Molecular therapy: the journal of the American Society of Gene Therapy 21, 629-637 (2013).
16. Hegde, M., et al. Combinational targeting offsets antigen escape and enhances effector functions of adoptively transferred T cells in glioblastoma. Molecular therapy: the journal of the American Society of Gene Therapy 21, 2087-2101 (2013).
17. Johnson, L. A., et al. Rational development and characterization of humanized anti-EGFR variant III chimeric antigen receptor T cells for glioblastoma. Science translational medicine 7, 275ra222 (2015).
18. Morgan, R. A., et al. Case report of a serious adverse event following the administration of T cells transduced with a chimeric antigen receptor recognizing ERBB2. Molecular therapy: the journal of the American Society of Gene Therapy 18, 843-851 (2010).
19. Ahmed, N., et al. Human Epidermal Growth Factor Receptor 2 (HER2) -Specific Chimeric Antigen Receptor-Modified T Cells for the Immunotherapy of HER2-Positive Sarcoma. Journal of clinical oncology: official journal of the American Society of Clinical Oncology (2015).
20. Scheurer, M. E., Bondy, M. L., Aldape, K. D., Albrecht, T. & El-Zein, R. Detection of human cytomegalovirus in different histological types of gliomas. Acta neuropathologica 116, 79-86 (2008).
21. Cobbs, C. S., et al. Human cytomegalovirus infection and expression in human malignant glioma. Cancer research 62, 3347-3350 (2002).
22. Lucas, K. G., Bao, L., Bruggeman, R., Dunham, K. & Specht, C. The detection of CMV pp65 and IE1 in glioblastoma multiforme. Journal of neuro-oncology 103, 231-238 (2011).
23. Mitchell, D. A., et al. Sensitive detection of human cytomegalovirus in tumors and peripheral blood of patients diagnosed with glioblastoma. Neuro-oncology 10, 10-18 (2008).
24. Ghazi, A., et al. Generation of polyclonal CMV-specific T cells for the adoptive immunotherapy of glioblastoma. Journal of immunotherapy 35, 159-168 (2012).
25. Wakefield, A., et al. Is CMV a target in pediatric glioblastoma? Expression of CMV proteins, pp65 and IE1-72 and CMV nucleic acids in a cohort of pediatric glioblastoma patients. Journal of Neuro-oncology 125, 307-315 (2015).
26. Leen, A. M., et al. Monoculture-derived T lymphocytes specific for multiple viruses expand and produce clinically relevant effects in immunocompromised individuals. Nature medicine 12, 1160-1166 (2006).
27. Ahmed, N., et al. Regression of experimental medulloblastoma following transfer of HER2-specific T cells. Cancer research 67, 5957-5964 (2007).
28. Bollard, C. M., Rooney, C. M. & Heslop, H. E. T-cell therapy in the treatment of post-transplant lymphoproliferative disease. Nature reviews. Clinical oncology 9, 510-519 (2012).
29. Hong, J. J., et al. Successful treatment of melanoma brain metastases with adoptive cell therapy. Clinical cancer research: an official journal of the American Association for Cancer Research 16, 4892-4898 (2010).
30. Schuessler, A., et al. Autologous T-cell therapy for cytomegalovirus as a consolidative treatment for recurrent glioblastoma. Cancer research 74, 3466-3476 (2014).
31. Rooney, C. M., et al. Infusion of cytotoxic T cells for the prevention and treatment of Epstein-Barr virus-induced lymphoma in allogeneic transplant recipients. Blood 92, 1549-1555 (1998).
32. Heslop, H. E., et al. Long-term outcome of EBV-specific T-cell infusions to prevent or treat EBVrelated lymphoproliferative disease in transplant recipients. Blood 115, 925-935 (2010).
33. Cooper, L. J., et al. Enhanced antilymphoma efficacy of CD19-redirected influenza MP1-specific CTLs by cotransfer of T cells modified to present influenzaMP1. Blood 105, 1622-1631 (2005).
34. Okada, H., et al. Immunotherapy response assessment in neuro-oncology: a report of the RANO working group. The Lancet. Oncology 16, e534-542 (2015).
35. Franceschi, E., et al. Post progression survival in glioblastoma: where are we? Journal of neurooncology 121, 399-404 (2015).
36. Taal, W., et al. Single-agent bevacizumab or lomustine versus a combination of bevacizumab plus lomustine in patients with recurrent glioblastoma (BELOB trial): a randomised controlled phase 2 trial. The Lancet. Oncology 15, 943-953 (2014).
37. Berghoff, A. S., et al. Programmed death ligand 1 expression and tumor-infiltrating lymphocytes in glioblastoma. Neuro-oncology 17, 1064-1075 (2015).
38. Nduom, E. K., et al. PD-L1 expression and prognostic impact in glioblastoma. Neuro-oncology 18, 195-205 (2016).
39. Wei, J., et al. Glioma-associated cancer-initiating cells induce immunosuppression. Clinical cancer research: an official journal of the American Association for Cancer Research 16, 461-473 (2010).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A polynucleotide that encodes a HER2-specific chimeric antigen receptor.

2. The polynucleotide of claim 1, wherein the chimeric antigen receptor comprises a transmembrane domain selected from the group consisting of CD3-zeta, CD28. CD8, 4-1BB, CTLA4, CD27, and a combination thereof.

3. The polynucleotide of claim 1, wherein the chimeric antigen receptor comprises no more than one costimulatory endodomain.

4. The polynucleotide of claim 1, wherein the chimeric antigen receptor comprises more than one costimulatory endodomain.

5. The polynucleotide of claim 1, wherein the chimeric antigen receptor comprises co-stimulatory molecule endodomains selected from the group consisting of CD28, CD27, 4-1BB, OX40 ICOS, Myd88, CD40, and a combination thereof.

6. The polynucleotide of claim 1, wherein the chimeric antigen receptor comprises a scFv specific for HER2 that is selected from the group consisting of trastuzmab, FRP5, scFv800E6, F5cys, pertuzumab and a combination thereof.

7. An expression vector comprising the polynucleotide of claim 1.

8. The vector of claim 7, wherein the vector is a viral vector.

9. The vector of claim 8, wherein the viral vector is a retroviral vector, lentiviral vector, adenoviral vector, or adeno-associated viral vector.

10. A cell comprising the expression vector of claim 7.

11. The cell of claim 10, wherein said cell is an immune cell.

12. The cell of claim 11, wherein the immune cell is a T cell, NK cell, or NKT cell.

13. The cell of claim 10, wherein the cell is specific for another antigen.

14. The cell of claim 13, wherein the antigen is a tumor antigen.

15. The cell of claim 13, wherein the cell is virus-specific.

16. The cell of claim 15, wherein the cells are pp65CMV-specific T cells, CMV-specific T cells, EBV-specific T cells, Varicella Virus-specific T cells, Influenza Virus-specific T cells and/or Adenovirus-specific T cells.

17. The cell of claim 10, wherein the cell comprises a chimeric antigen receptor other than the HER2-specific chimeric antigen receptor.

18. A method of treating an individual for cancer, comprising the step of providing to the individual a therapeutically effective amount of a plurality of any of the cells of claim 10 or a substrate comprising a HER2 chimeric antigen receptor

19. The method of claim 18, wherein the cancer is HER2 positive.

20. The method of claim 18, wherein the cancer is refractory or recurrent.

21. The method of claim 18, wherein the cancer is sarcoma or glioblastoma.

22. The method of claim 21, wherein the sarcoma is osteosarcoma.

23. The method of claim 18, wherein the therapeutically effective amount of a plurality of the cells is at a dose of at least 1×10⁴/m², 1×10⁵/m², 1×10⁶/m², 1×10⁷/m², 1×10⁸/m², 1×10⁹/m², or 1×10¹⁰/m².

24. The method of claim 18, wherein the therapeutically effective amount of a plurality of, the cells is at a dose of no more than 1×10¹⁰/m², 1×10⁹/m², 1×10⁸/m², 1×10⁷/m², 1×10⁶/m², 1×10⁵/m², or 1×10⁴/m².

25. The method of claim 18, wherein the cell is an immune cell that transgenically expresses one or more chemokine receptors.

26. The method of claim 25, wherein the chemokine receptor is a receptor for a chemokine expressed by the cancer.

27. The method of claim 25, wherein the chemokine is CXCL1, CXCL8, CCL2, and/or CCL17.

28. The method of claim 18, wherein the individual is provided a therapeutically effective amount of an additional cancer therapy.

29. The method of claim 28, wherein the additional cancer therapy is given to the individual before, during, and/or after the individual is given the plurality of cells.

30. The method of claim 28, wherein the additional therapy comprises surgery, drug therapy, chemotherapy, radiation, immunotherapy, or a combination thereof.

31. The method of claim 18, wherein the individual is given lymphodepleting therapy prior to being given the plurality of cells.

32. The method of claim 18, wherein the individual is not given lymphodepleting therapy prior to being given the plurality of cells.

33. The method of claim 30, wherein the immunotherapy comprises one or more checkpoint antibodies.

34. The method of claim 33, wherein the checkpoint antibodies recognize CTLA4, PD-1, PD-L1, TIM3, BLTA, VISTA and/or LAG3.

35. The method of claim 18, wherein the cell comprises an inhibitory receptor.

36. The method of claim 18, wherein the method occurs without the administration of one or more cytokines and without lymphodepleting therapy and occurs with a cell dose in the range of 1×10⁴/m² to 1×10¹⁰/m².

37. The method of claim 18, wherein the method comprises the administration of one or more cytokines and comprises the step of providing lymphodepleting therapy to the individual and occurs with a cell dose in the range of 1×10⁴/m² to 1×10¹⁰/m².

38. The method of claim 36, wherein the cytokine is IL2, IL7, IL12, and/or IL15.

39. The method of claim 18, wherein the cells are provided to the individual by a route that is parenteral, transdermal, intraluminal, intra-arterial, intrathecal, intravenous, subcutaneous, intraperitoneal, intramuscular, topical, intradermal, by infusion, by injection, or a combination thereof.

40. A kit, comprising the polynucleotide of claim 1, the expression vector of claim 7, and/or the cells of claim 10, wherein the polynucleotide, expression vector, and or cells are housed in a suitable container.
